(19) United States
(12) Patent Application Publication
(10) Pub. No.: US 2026/0077356 A1
(43) Pub. Date: Mar. 19, 2026

(54) MACHINE LEARNING METHODS TO CONTROL MICROBUBBLE DYNAMICS

(71) Applicant: Georgia Tech Research Corporation, Atlanta, GA (US)

(72) Inventors: Hohyun Lee, Atlanta, GA (US); Costas Arvanitis, Atlanta, GA (US); Victor Menezes, Atlanta, GA (US)

(21) Appl. No.: 19/327,470

(22) Filed: Sep. 12, 2025

Related U.S. Application Data
(60) Provisional application No. 63/694,394, filed on Sep. 13, 2024.

(51) Int. Cl. B01L 3/00; G16H 20/40

(52) U.S. Cl. CPC B01L 3/502769; G16H 20/40; B01L 2200/143; B01L 2400/0436

(57) ABSTRACT

Systems and methods for predicting and controlling bubble dynamics. The controller may comprise one or more processors. The controller may also comprise a machine learning model trained based on bubble acoustic emission data. The controller may also comprise at least one memory in communication with the controller and the machine learning model and storing computer program code. The computer program code may cause the controller to monitor acoustic emission levels of one or more bubbles in a body of a subject. The computer program code may further cause the controller to predict at least one acoustic emission level indicative of a collapse of the one or more bubbles. The computer program code may also cause the controller to cause an acoustic transducer to emit acoustic energy based at least in part on the at least one predicted acoustic emission level indicative of the collapse of the one or more bubbles.

### CROSS-REFERENCE TO RELATED APPLICATIONS

This application claims the priority benefit of U.S. Provisional Patent Application Ser. No. 63/694,394, filed Sep. 13, 2024, which is hereby incorporated by reference in its entirety.

### STATEMENT REGARDING FEDERALLY SPONSORED RESEARCH OR DEVELOPMENT

This invention was made with government support under Agreement No. CA239039, awarded by the National Institutes of Health. The government has certain rights in the invention.

### BACKGROUND OF THE DISCLOSURE

### 1. Field of the Invention

The present invention relates generally to systems and methods, and more particularly to focused ultrasound (FUS) systems combined with microbubble (MB) ultrasound systems and methods thereof.

### 2. Description of Related Art

Ultrasound has emerged as a novel modality for the treatment and imaging of many conditions, including brain diseases and disorders. When enhanced by circulating microbubble contrast agents, for example lipid, albumin, or polymer-shelled gas pockets that scatter sound and vibrate in response to incident ultrasound, ultrasound can enable a range of new therapeutic interventions and open new possibilities for imaging. Current ultrasound systems, however, are limited in both imaging and targeting.

Microbubbles (MBs) are ultrasound contrast agents (USCA) that are widely used for ultrasound (US) imaging in tissues. MBs' contrasting ability comes from their i) non-linear oscillatory behavior and ii) high resonant but linear scattering cross section, which ultimately enhances US image quality. Apart from their contributions in US imaging, recent studies also discovered the therapeutic potential of MBs when combined with focused ultrasound (FUS). This therapeutic strategy, known as MB-FUS, is an emerging technology that provides a physical method to reversibly increase the permeability of the blood-brain barrier (BBB), which is a major obstacle in delivery of therapeutic agents to the brain.

MB acoustic emissions (AE), which are the radiated pressures from oscillating MBs, can provide reasonably accurate inferences about the strength and type of MB dynamics and hence, the MB-FUS treatment outcome. Current MB dynamics monitoring strategies rely on AE detection, either with single or multiple element passive cavitation detectors (PCD). In addition to monitoring purpose, AE nonlinearly (but positively) correlates with excitation pressure, which also provides control opportunity. Consequently, the current state of art of controlling MB dynamics during MB-FUS is based on implementation of open-loop (pressure increments by preset amount) or closed-loop (adaptable pressure increments) control algorithms onto harmonic levels of AE. Machine learning (ML) is a well-established statistical method with potential to be integrated with control of complex mechanisms to improve accuracy and robustness. Current applications of machine learning (ML) on field of FUS are only focused on imaging, treatment outcome predictions, or acoustic field reconstructions rather than real-time MB-FUS therapy.

Therefore, there is a need for systems and methods that resolve the problems with machine learning algorithms used with MB-FUS controller that overcome current limitations.

### SUMMARY OF THE INVENTION

Briefly described, according to exemplary embodiments of the present invention, systems, and methods of an innovative system for predicting and controlling bubble dynamics. In some embodiments, the present inventions predict and determine at least one dynamic property of the one or more microbubbles in real-time.

In an exemplary embodiment of the present disclosure, a controller may comprise one or more processors. In various embodiments, the controller may further comprise at least one machine learning model. The at least one machine learning model may be in communication with the controller and/or be trained based at least in part on bubble acoustic emission data. In various embodiments, the controller may also comprise at least one memory in communication with the controller and/or the machine learning model. The at least one memory may store computer program code and may be executed by the controller. The computer program code may cause the controller to monitor acoustic emission levels of one or more bubbles in a body of a subject. The computer program code may further cause the controller to predict, via the machine learning model, at least one acoustic emission level indicative of a collapse of the one or more bubbles. The computer program code may also cause the controller to cause, in real-time, an acoustic transducer to emit acoustic energy to the one or more bubbles based at least in part on the at least one predicted acoustic emission level indicative of the collapse of the one or more bubbles.

In various embodiments, the controller may be one or more of a constant pressure sonication controller, an open-loop controller configured to adjust acoustic energy by a preset control law, a reactive controller configured to adjust acoustic energy upon detection of the collapse of the one or more bubbles, a closed-loop controller configured to dynamically adjust acoustic energy based on real-time feedback, or a combination thereof. In various embodiments, the machine learning model may be one or more of a classification model, a regression model, a support vector machine model, a logistic regression model, a different neural network model, a deep learning model, a multi-layer perceptron model, or a combination thereof. In various embodiments, the one or more bubbles may be one or more of microbubbles, ultrasound contrast microbubbles, nanobubbles, cavitation nuclei, bubbles generated in situ during focused ultrasound (FUS) intervention, or a combination thereof.

In various embodiments, the bubbles acoustic emission data may comprise at least one feature arranged in a matrix. In various embodiments, the at least one feature may be arranged in the matrix comprises one or more of 2^{nd}-8^{th} harmonic levels, one or more of 2^{nd}-8^{th} ultra-harmonic levels, presence of at least one tumor, bubble kinetic level derived from normalized temporal harmonic level change, pulse number during sonication, at least one pressure, at least one presence of a disease, or a combination thereof.

In various embodiments, preventing the collapse of the one or more bubbles may prevent damage to at least a portion of at least one vessel and/or at least one tissue of the subject.

In various embodiments, an emitted acoustic energy may be maintained below the predicted acoustic emission level that would cause the one or more bubbles to collapse. In various embodiments, adjusting the acoustic energy may comprise adjusting one or more of a peak negative pressure, an acoustic intensity, a pulse repetition frequency (PRF), a pulse duration, a pulse length, a duty cycle, a number of pulses, a waveform shape, or a combination thereof.

In various embodiments, the at least one memory may further comprise computer program code that, when executed by the controller, may cause the controller to generate at least one output based on a prediction of the machine learning model. In various embodiments, the at least one output may be a real-time advisory instruction configured to guide therapeutic sonication operated at a constant pressure. In various embodiments, the at least one output may be a real-time advisory instruction configured to adjust acoustic energy delivered by an acoustic transducer operated with controller.

In various embodiments, predicting and controlling bubble dynamics may be used for one or more of ultrasound imaging, therapeutic treatment, therapeutic treatment for a tumor, liquid biopsy, drug delivery, increasing a permeability of a blood-brain barrier, maximizing treatment efficiency, histotripsy, gene delivery, other FUS-related inventions including but not limited to neuromodulation, ablation, or immunomodulation, or a combination thereof.

In various embodiments, the at least one memory may further comprise computer program code that, when executed by the controller, may cause the controller to determine, using the machine learning model, one or more indicators of a collapse of the one or more bubbles. In various embodiments, the one or more indicators is one or more of at least one bubble kinetic, an average pressure, a maximum pressure, a minimum pressure, at least one microbubble kinetic, a brain region, presence of at least one tumor, or a combination thereof, wherein the one or more indicators indicates an acoustic emission level indicative of the collapse of the one or more bubbles.

In another exemplary embodiment of the present disclosure a non-transitory computer readable medium having stored thereon instructions is provided for predicting and controlling of bubble dynamics. In various embodiments, the non-transitory computer readable medium may be in communication with an acoustic transducer. In various embodiments, the instructions may cause the processor to monitor acoustic emission levels of one or more bubbles in a body of a subject. In various embodiments, the instructions may further cause the processor to predict an acoustic emission level indicative of a collapse of one or more. In various embodiments, the instructions may also cause the processor to cause, in real-time, the acoustic transducer to emit acoustic energy to the one or more bubbles based at least in part on the predicted acoustic emission level indictive of the collapse of the one or more bubbles.

In various embodiments, the non-transitory computer readable medium may be a portion of a controller. In various embodiments, the controller may be one or more of a constant pressure sonication controller, an open-loop controller configured to adjust acoustic energy by a preset control law, a reactive controller configured to adjust acoustic energy upon detection of the collapse of the one or more bubbles, a closed-loop controller configured to dynamically adjust acoustic energy based on real-time feedback, or a combination thereof. In various embodiments, the one or more bubbles may be one or more of microbubbles, ultrasound contrast microbubbles, nanobubbles, cavitation nuclei, bubbles generated in situ during focused ultrasound (FUS) intervention, or a combination thereof.

In various embodiments, bubbles acoustic emission data may comprise at least one feature arranged in a matrix. In various embodiments, the at least one feature arranged in the matrix may comprise one or more of 2^{nd}-8^{th} harmonic levels, one or more of 2^{nd}-8^{th} ultra-harmonic levels, presence of at least one tumor, microbubble kinetic level derived from normalized temporal harmonic level change, pulse number during sonication, at least one pressure, at least one presence of a disease, or a combination thereof.

In various embodiments, preventing the collapse of the one or more bubbles may prevent damage to at least a portion of at least one vessel and/or at least one tissue of the subject.

In various embodiments, an emitted acoustic energy may be maintained below the predicted acoustic emission level that would cause the one or more bubbles to collapse. In various embodiments, adjusting the acoustic energy may comprise adjusting one or more of a peak negative pressure, an acoustic intensity, a pulse repetition frequency (PRF), a pulse duration, a pulse length, a duty cycle, a number of pulses, a waveform shape, or a combination thereof.

In various embodiments, the non-transitory computer readable medium may further comprise instructions, which when executed by one or more processors, may further cause the processors to generate at least one output based on at least one prediction from a machine learning model. In various embodiments, the at least one output may be a real-time advisory instruction configured to guide therapeutic sonication operated at a constant pressure. In various embodiments, the at least one output may be a real-time advisory instruction configured to adjust acoustic energy delivered by an acoustic transducer operated with controller.

In various embodiments, wherein predicting and controlling bubble dynamics may be used for one or more of ultrasound imaging, therapeutic treatment, therapeutic treatment for a tumor, liquid biopsy, drug delivery, increasing a permeability of a blood-brain barrier, maximizing treatment efficiency, histotripsy, gene delivery, other FUS-related inventions including but not limited to neuromodulation, ablation, or immunomodulation, or a combination thereof.

In various embodiments, the non-transitory computer readable medium may further comprise instructions, which when executed by one or more processors, may further cause the processors to determine one or more indicators of a collapse of the one or more bubbles. In various embodiments, the one or more indicators is one or more of at least one bubble kinetic, an average pressure, a maximum pressure, a minimum pressure, at least one microbubble kinetic, a brain region, presence of at least one tumor, or a combination thereof, wherein the one or more indicators indicates at least one acoustic emission level indicative of the collapse of the one or more bubbles.

In yet another exemplary embodiment of the present disclosure a method is provided for controlling bubble dynamics. In various embodiments, the method may comprise monitoring acoustic emission levels of one or more bubbles in a body of a subject. In various embodiments, the method may further comprise predicting, via a machine learning model, at least one acoustic emission level that would indicate a collapse of the one or more bubbles. In various embodiments, the method may also comprise emitting an acoustic energy to the one or more bubbles based at least in part on the predicted acoustic emission level that would indicate the collapse of the one or more bubbles. The one or more bubbles may be one or more of microbubbles, ultrasound contrast microbubbles, nanobubbles, cavitation nuclei, bubbles generated in situ during focused ultrasound (FUS) intervention, or a combination thereof.

In various embodiments, the method may further comprise training the machine learning model based at least on bubble acoustic emission data. In various embodiments, the machine learning model may be one or more of a classification model, a regression model, a support vector machine model, a logistic regression model, a different neural network model, a deep learning model, a multi-layer perceptron model, or a combination thereof. In various embodiments, the bubbles acoustic emission data may comprise at least one feature arranged in a matrix. In various embodiments, the at least one feature arranged in the matrix may comprise one or more of 2^{nd}-8^{th} harmonic levels, 2^{nd}-8^{th} ultra-harmonic levels, presence of at least one tumor, microbubble kinetic level derived from normalized temporal harmonic level change, pulse number during sonication, at least one pressure, at least one presence of a disease, or a combination thereof.

In various embodiments, the method may further comprise determining, using the machine learning model, one or more indicators of a collapse of the one or more bubbles. The one or more indicators may be one or more of at least one bubble kinetic, an average pressure, a maximum pressure, a minimum pressure, at least one microbubble kinetic, a brain region, presence of at least one tumor, or a combination thereof, wherein the one or more indicators indicates at least one acoustic emission level indicative of the collapse of the one or more bubbles. In various embodiments, the method may also comprise generating at least one output based at least in part on at least one prediction from a machine learning model. In various embodiments, the at least one output may be a real-time advisory instruction configured to guide therapeutic sonication operated at a constant pressure. In various embodiments, the at least one output may be a real-time advisory instruction configured to adjust acoustic energy delivered by an acoustic transducer operated with controller.

In various embodiments, controlling the bubble dynamics may use one or more of a constant pressure sonication controller, an open-loop controller configured to adjust acoustic energy by a preset control law, a reactive controller configured to adjust acoustic energy upon detection of the collapse of the one or more bubbles, a closed-loop controller configured to dynamically adjust acoustic energy based on real-time feedback, or a combination thereof.

These and other aspects, features, and benefits of the claimed invention(s) will become apparent from the following detailed written description of the preferred embodiments and aspects taken in conjunction with the following drawings, although variations and modifications thereto may be affected without departing from the spirit and scope of the novel concepts of the disclosure.

### BRIEF DESCRIPTION OF THE DRAWINGS

Implementations, features, and aspects of the disclosed technology are described in detail herein and are considered a part of the claimed disclosed technology. Other implementations, features, and aspects can be understood with reference to the following detailed description, accompanying drawings, and claims. Wherever possible, the same reference numbers are used throughout the drawings to refer to the same or like members of an embodiment. Reference will now be made to the accompanying figures and flow diagrams, which are not necessarily drawn to scale.

FIG. 1 illustrates an exemplary schematic of a controller in accordance with various embodiments of the present disclosure.

FIG. 2 illustrates an exemplary schematic of a system in accordance with various embodiments of the present disclosure.

FIG. 3 illustrates an exemplary schematic for gathering a training data in accordance with various embodiments of the present disclosure.

FIG. 4 graphically illustrates the accuracy for predicting at least one bubble collapse using the trained data set and machine learning model in accordance with various embodiments of the present disclosure.

FIG. 5A graphically illustrates SHAP analysis on an exemplary training dataset in accordance with various embodiments of the present disclosure.

FIG. 5B graphically illustrates an exemplary overall importance of features affecting machine learning (ML) model output in accordance with various embodiments of the present disclosure.

FIG. 6 illustrates an exemplary schematic of a machine learning model in communication with a controller in accordance with various embodiments of the present disclosure.

FIG. 7A graphically illustrates a cavitation threshold model acquired using an exemplary training dataset in accordance with various embodiments of the present disclosure.

FIG. 7B graphically illustrates exemplary pressure decisions by a controller in accordance with various embodiments of the present disclosure.

FIG. 7C graphically illustrates an exemplary control performance for attaining harmonic emission in accordance with various embodiments of the present disclosure.

FIG. 8A graphically illustrates exemplary pressure decisions by a controller in accordance with various embodiments of the present disclosure.

FIG. 8B graphically illustrates an exemplary histogram for an acoustic emission level that would represent one or more bubbles to collapse in accordance with various embodiments of the present disclosure.

FIG. 8C graphically illustrates an exemplary model output for conventional controllers in accordance with various embodiments of the present disclosure.

FIG. 8D graphically illustrates exemplary dynamic contrast-enhanced magnetic resonance imaging (DCE-MRI) permeability data in accordance with various embodiments of the present disclosure.

FIG. 9A illustrates exemplary target locations in accordance with various embodiments of the present disclosure.

FIG. 9B graphically illustrates an exemplary 7^{th} harmonic emission for each controller in accordance with various embodiments of the present disclosure.

FIG. 9C graphically illustrates an exemplary histogram for a 7^{th} harmonic emission in accordance with various embodiments of the present disclosure.

FIG. 9D graphically illustrates an exemplary histogram for an acoustic emission level that would cause the one or more bubbles to collapse in accordance with various embodiments of the present disclosure.

FIG. 9E graphically illustrates an exemplary histogram for a machine learning model in communication with a controller in accordance with various embodiments of the present disclosure.

FIG. 9F illustrates exemplary MRI images using an example controller in accordance with various embodiments of the present disclosure.

FIG. 9G graphically illustrates an exemplary quantification of Kₜᵣₐₙₛ values through DCE-MRI in accordance with various embodiments of the present disclosure.

FIG. 10 illustrates a flow diagram of an exemplary method in accordance with various embodiments of the present disclosure.

### DETAILED DESCRIPTION

Although certain embodiments of the disclosure are explained in detail, it is to be understood that other embodiments are contemplated. Accordingly, it is not intended that the disclosure is limited in its scope to the details of construction and arrangement of components set forth in the following description or illustrated in the drawings. Other embodiments of the disclosure are capable of being practiced or carried out in various ways. Also, in describing the embodiments, specific terminology will be resorted to for the sake of clarity. It is intended that each term contemplates its broadest meaning as understood by those skilled in the art and includes all technical equivalents which operate in a similar manner to accomplish a similar purpose.

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

Also, in describing the preferred exemplary embodiments, terminology will be resorted to for the sake of clarity. It is intended that each term contemplates its broadest meaning as understood by those skilled in the art and includes all technical equivalents that operate in a similar manner to accomplish a similar purpose.

As used in the specification and the appended claims, the singular forms “a,” “an” and “the” include plural referents unless the context clearly dictates otherwise. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

By “comprising” or “containing” or “including” is meant that at least the named element, system, compound, member, particle, or method step is present in the composition or element or system or article or method, but does not exclude the presence of other compounds, materials, particles, method steps, even if the other such compounds, material, particles, method steps have the same function as what is named.

As used here, the term “acoustic energy” includes one or more sonication parameters that may be adjusted to influence bubbles. Acoustic energy is not limited to pressure magnitude but may also include, without limitation, one or more of a peak negative pressure, an acoustic intensity, a pulse repetition frequency (PRF), a pulse duration, a pulse length, a duty cycle, a number of pulses, a waveform shape, or a combinations thereof. In various embodiments, one or more of the machine learning model, the controller, the system, or a combination thereof may adjust at least one of these parameters individually or in combination to predict inertial cavitation and optimize therapeutic effect.

As used here, the use of terms such as “bubbles” or “microbubbles” is intended to broadly encompass any type of bubble responsive to acoustic energy, including but not limited to, lipid-, albumin-, or polymer-shelled contrast agent microbubbles, gas vesicles, nanobubbles, phase-change droplets, cavitation nuclei, and bubbles generated from other FUS interventions.

As used herein, the term “controller” broadly refers to one or more of mechanisms, hardware, software, or a combination thereof, configured to influence sonication conditions. A controller is not limited to closed-loop systems, but encompasses constant pressure sonications, open-loop controllers (e.g., constant control law with preset increments), and those controllers that adjust parameters upon bubble collapse, and closed-loop controllers that dynamically adjust based on real-time feedback. In various embodiments, the machine learning model may provide predictive and/or advisory inputs to any of these controller types.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

Mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

To facilitate an understanding of the principles and features of the disclosure, various illustrative embodiments are explained below. In a non-limiting embodiments, the presently disclosed subject matter is described in the context of systems and methods for imaging and focusing using ultrasound and, more particularly, to estimation of dynamic properties of one or more bubbles in a focused ultrasound systems. In some examples, the systems and methods may be described in the context of treating patients, including human and/or other animal patients. The present disclosure, however, is not so limited and can be applicable in outer contexts. For example, some examples of the present disclosure may improve upon imaging and targeting in inanimate systems. Additionally, reference is made herein to ultrasounds techniques for targeting through bone, including a human skull. It will be understood that such disclosure is illustrative, as the imaging and focusing techniques can be applied equally to other surfaces. Accordingly, when the present disclosure is described in the context of estimating dynamic properties of the one or more bubbles in focused ultrasound systems in any particular biological setting, it will be understood that other embodiments can take the place of those referred to.

In various embodiments, the machine learning (ML) model as described herein may be configured to assist with predicting and/or controlling the dynamics of the one or more bubbles. In various embodiments, the one or more bubbles may be one or more of microbubbles (MB), ultrasound contrast microbubbles, nanobubbles, cavitation nuclei, bubbles generated in situ during focused ultrasound (FUS) intervention, lipid-, albumin-, or polymer-shelled contrast agent microbubbles, gas vesicles, phase-change droplets, or a combination thereof. The ML model may predict the collapse of at least one bubble during one or more of ultrasound imaging, therapeutic treatment, therapeutic treatment for a tumor, liquid biopsy, drug delivery, increasing a permeability of a blood-brain barrier, maximizing treatment efficiency, histotripsy, gene delivery, other FUS-related inventions including but not limited to neuromodulation, ablation, or immunomodulation, or a combination thereof. The ML model may predict the collapse of at least one bubble and/or control acoustic energy emitted in order to prevent damage to at least a portion of at least one vessel and/or at least one tissue of the subject from occurring. In various embodiments, two or more ML models may be used to predict and/or control bubbles dynamics. In one or more embodiments, a first ML model may be configured to predict at least one acoustic emission level that may be indicative of the collapse of one or more bubbles and/or one or more additional ML models may receive a prediction from the first ML model and may assist with controlling (e.g., adjusting emitted acoustic energy) emitted acoustic energy.

In various embodiments, the ML model (e.g., the first ML model and/or the one or more additional ML models) may be trained by at least one training dataset that comprises information relating to the onset of broadband emission (i.e., indicative of a collapse of one or more bubbles) and/or harmonic emission (i.e., usual indication of bubble dynamics). In various embodiments, one or more of the following features can be regarded as important: i) bubbles' temporal kinetics in the body (e.g., microbubbles' temporal kinetics in the body), ii) target region in the brain (different region has different vasculature and thus different number of M), iii) presence of disease (e.g., tumour's abnormal vasculature affects MB dynamics and number of bubbles), and iv) spectral content such as harmonic and ultra-harmonic emission from AE. As described herein, the ML model may be in communication with at least one controller.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

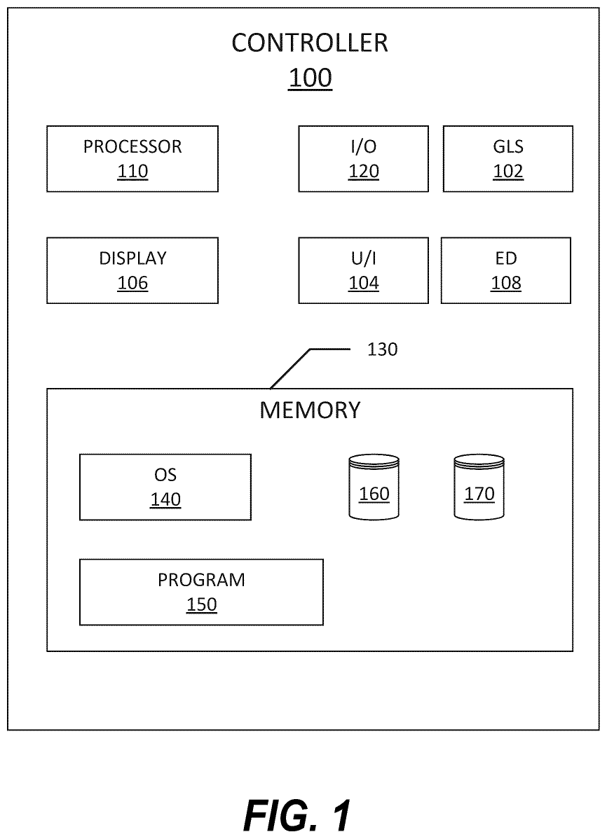

FIG. 1 illustrates an exemplary schematic of a controller in accordance with various embodiments of the present disclosure. In various embodiments, the controller 100 may be used in a focused ultrasound (FUS) environment and/or with one or more bubbles. In other embodiments, the controller may be used in one or more of ultrasound imaging, a therapeutic treatment, a therapeutic treatment for a tumor, a liquid biopsy, a drug delivery, increasing a permeability of a blood-brain barrier, maximizing treatment efficiency, histotripsy, gene delivery, other FUS-related inventions including but not limited to neuromodulation, ablation, or immunomodulation, or a combination thereof. In various embodiments, the one or more bubbles may be one or more of microbubbles, ultrasound contrast microbubbles, nanobubbles, cavitation nuclei, bubbles generated in situ during focused ultrasound (FUS) intervention, or a combination thereof.

The components and arrangements shown in FIG. 1 are not intended to limit the disclosed embodiments as the components used to implement the disclosed processes and features may vary. As non-limiting examples, the controller 100 may be one or more of a cell phone, a smart phone, a tablet computer, a laptop computer, a desktop computer, a sever, or another electronic device. In one or more embodiments, the controller 100 may be one or more of a constant pressure sonication controller, an open-loop controller configured to adjust acoustic energy by a preset control law, a reactive controller configured to adjust acoustic energy upon detection of at least one bubble collapse, a closed-loop controller configured to dynamically adjust acoustic energy based on real-time feedback, or a combination thereof.

As shown in FIG. 1, the controller 100 may include one or more processors 110, one or more input/output (“I/O”) devices 120, and/or one or more memories 130 containing an operating system (“OS”) 140 and a program 150. In one or more embodiments, the one or more memories 130 may further contain at least one training dataset 160 and/or at least one machine learning (ML) model 170. In other embodiments, the at least one training data set 160 and/or the at least one ML model 170 may be separate from the controller (not depicted) such that, the at least one training data set 160 and/or the at least one ML model 170 may be in communication with the controller 100.

In various embodiments, the controller 100 may further include one or more geographic location sensors (“GLS”) 102 for determining the geographic location of controller 100, one or more displays 106 for displaying content such as text messages, items, and selectable buttons/icons/links, one or more environmental data (“ED”) sensors 108 for obtaining environmental data including audio and/or visual information, and/or one or more user interface (“U/I”) devices 104 for receiving user input data, such as data representative of a click, a scroll, a tap, a press, or typing on an input device that can detect tactile inputs. User input data may also be non-tactile inputs that may be otherwise detected by ED sensor 108. For example, user input data may include auditory commands. In some embodiments, environmental data sensor 108 may include a microphone and/or an image capture device, such as a digital camera.

Processor 110 may be one or more known processing devices, such as a microprocessor from the Pentium™ family manufactured by Intel™ or the Turion™ family manufactured by AMD™. Processor 110 may constitute a single core or multiple core processor that executes parallel processes simultaneously. Processor 110 may be a single core processor, for example, that is configured with virtual processing technologies. In certain embodiments, processor 110 may use logical processors to simultaneously execute and control multiple processes. Processor 110 may implement virtual machine technologies, or other similar known technologies to provide the ability to execute, control, run, manipulate, store, etc. multiple software processes, applications, programs, etc. One of ordinary skill in the art would understand that other types of processor arrangements could be implemented that provide for the capabilities disclosed herein.

Memory 130 may include one or more memory devices that store data and instructions used to perform one or more features of the disclosed embodiments. Memory 130 may include, in some implementations, one or more suitable types of memory (e.g. such as volatile or non-volatile memory, a random access memory (RAM), a read only memory (ROM), a programmable read-only memory (PROM), an erasable programmable read-only memory (EPROM), an electrically erasable programmable read-only memory (EEPROM), one or more magnetic disks, one or more optical disks, one or more floppy disks, one or more hard disks, one or more removable cartridges, a flash memory, a redundant array of independent disks (RAID), and the like), for storing files including an operating system, one or more application programs (including, for example, a web browser application, a widget or gadget engine, and or other applications, as necessary), executable instructions and data. In one embodiment, the processing techniques described herein are implemented as a combination of executable instructions and data within memory 130.

Memory 130 may also include any combination of one or more databases controlled by memory controller devices (e.g., server(s), etc.) or software, such as document management systems, Microsoft™ SQL databases, SharePoint™ databases, Oracle™ databases, Sybase™ databases, or other relational databases. Memory 130 may include software components that, when executed by processor 110, perform one or more processes consistent with the disclosed embodiments. In some embodiments, memory 130 may include at least one training dataset 160 for storing related data to enable the controller 100 to perform one or more of the processes and functionalities associated with the disclosed embodiments.

The controller 100 may include one or more storage devices configured to store information used by processor 110 (or other components) to perform certain functions related to the disclosed embodiments. In one example, the controller 100 may include memory 130 that includes instructions to enable processor 110 to execute one or more applications, such as server applications, network communication processes, and any other type of application or software known to be available on computer systems. Alternatively, the instructions, application programs, etc. may be stored in an external storage or available from a memory over a network. The one or more storage devices may be a volatile or non-volatile, magnetic, semiconductor, tape, optical, removable, non-removable, or other type of storage device or tangible computer-readable medium.

In one embodiment, the controller 100 may include memory 130 that includes instructions that, when executed by processor 110, perform one or more processes consistent with the functionalities disclosed herein. Methods, systems, and articles of manufacture consistent with disclosed embodiments are not limited to separate programs or computers configured to perform dedicated tasks. The controller 100 may include memory 130 including one or more programs 150, for example, to perform one or more functions of the disclosed embodiments. Moreover, processor 110 may execute one or more programs 150 located remotely from the controller 100. For example, the controller 100 may access one or more remote programs 150, that, when executed, perform functions related to disclosed embodiments.

A mobile network interface (not depicted) may provide access to a cellular network, the Internet, or another wide-area network. In some embodiments, a mobile network interface may include hardware, firmware, and/or software that allows processor(s) 110 to communicate with other devices via wired or wireless networks, whether local or wide area, private or public, as known in the art. A power source (not depicted) may be configured to provide an appropriate alternating current (AC) or direct current (DC) to power components.

As described above, the controller 100 may be configured to remotely communicate with one or more other devices, such as user device, machine learning model, neural network, and/or other external devices. According to some embodiments, the controller 100 may utilize machine learning model (or other suitable logic) to predict at least one future collapse of at least one bubble.

The controller 100 may also include one or more I/O devices 120 that may include one or more interfaces (e.g., transceivers) for receiving signals or input from devices and providing signals or output to one or more devices that allow data to be received and/or transmitted by the controller 100. The controller 100 may include interface components, for example, which may provide interfaces to one or more input devices, such as one or more keyboards, mouse devices, touch screens, track pads, trackballs, scroll wheels, digital cameras, microphones, sensors, and the like, that enable the computing device 120 to receive data from one or more users.

While the controller 100 has been described as one form for implementing the techniques described hereinafter, those having ordinary skill in the art will appreciate that other, functionally equivalent techniques may be employed. As is known in the art, some or all of the functionality implemented via executable instructions may also be implemented using firmware and/or hardware devices such as, for example, application specific integrated circuits (ASICs), programmable logic arrays, state machines, etc. Furthermore, other implementations of the computing device may include a greater or lesser number of components than those illustrated.

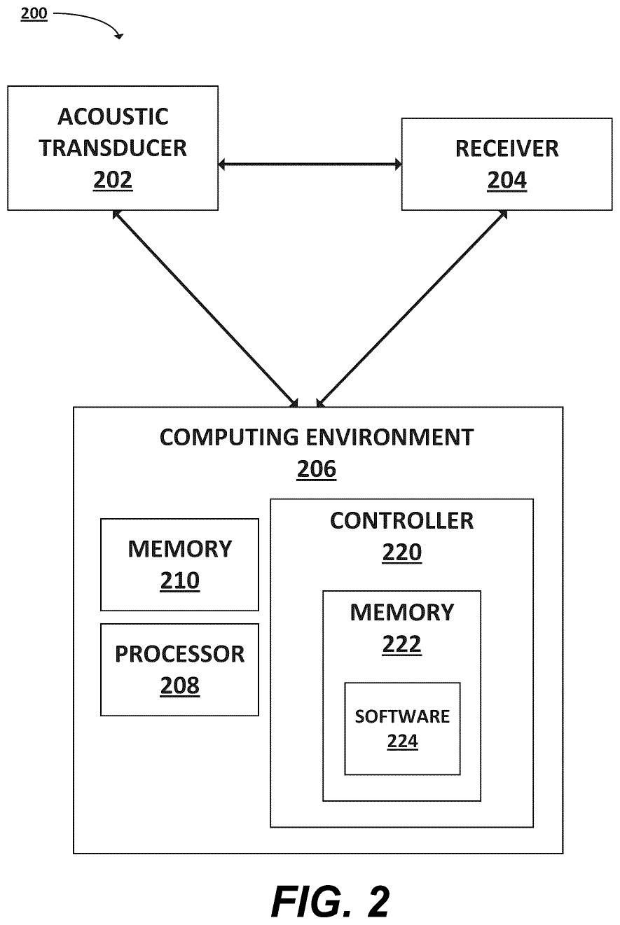

FIG. 2 illustrates an exemplary system 200 that may implement certain aspects of the present disclosure. The components and/or arrangements shown in FIG. 2 are not intended to limit the disclosed embodiments as the components used to implement the disclosed processes and features may vary. As shown in FIG. 2, in some implementations the system 200 can include at least one acoustic transducer 202, at least one receiver 204, and/or at least one computing environment 206 (e.g., controller, described above, and/or at least one ML model). The at least one computing environment 206 may include one or more processors 208, one or more transceivers, a user portal, and/or one or more controllers 220. In various embodiments, the one or more controller 220 may comprise one or more of the same components described in reference to FIG. 1. In various embodiments, the one or more controllers 220 may be a separate component from the computing environment 206, such that, the one or more controllers 220 may be in communication with the at least one transducer 202, at least one receiver 204, and/or at least one computing environment 206. In various embodiments, the at least one transducer 202 may be in communication with the one or more controllers 220, at least one receiver 204, at least one computing environment 206, and/or at least one ML model.

In various embodiments, the at least one acoustic transducer 202 can be a device that produces sound waves across a surface and to a region of interest, the at least one receiver 206 (e.g., sensor) can receive the radio frequency (RF) signals of the ultrasound waves bouncing off of a scatterer within the region of interest. In some examples, the at least one acoustic transducer 202 and the at least one receiver 204 can be a single piece of equipment that both transmits ultrasound waves and receives RF signals. In one or more embodiments, the at least one acoustic transducer 202 can adjust one or more of a peak negative pressure, an acoustic intensity, a pulse repetition frequency (PRF), a pulse duration, a pulse length, a duty cycle, a number of pulses, a waveform shape, or a combination thereof.

The at least one acoustic transducer 202 may be one or more of a linear array transducer, a curved array transducer, a phased array transducer, an endocavitary transducer, a 3D transducer, a 4D transducer, an intravascular ultrasound transducer, a focused ultrasound transducer, any other transducer capable of performing the desired function, or a combination thereof. The receiver 204 may be one or more of an analog receiver, a digital receiver, a beamforming receiver, a dynamic range receiver, a time-gain compensation receiver, a doppler receiver, a harmonic imaging receiver, any other transducer capable of performing the desired function, or a combination thereof. In various embodiments, the at least one acoustic transducer may emit acoustic energy configured to be controlled by the controller and/or ML model. The emitted acoustic energy may be directed at least partially towards the one or more bubbles and/or adjust one or more of a peak negative pressure, an acoustic intensity, a pulse repetition frequency (PRF), a pulse duration, a pulse length, a duty cycle, a number of pulses, a waveform shape, or a combination thereof.

In various embodiments, the at least one acoustic transducer 202, at least one receiver 204, at least one computing environment 206, and/or at least one controller 220 may be configured to communicate over a network, such as a local area network (LAN), Wi-Fi, Bluetooth, or other type of network, with the at least one transceiver and/or computing environment and may be connected to an intranet or the Internet, among other things. In various embodiments, the at least one computing environments 206 and/or at least one controller 220 may include one or more physical or logical devices (e.g., servers) or drives and may be implemented as a single server or a bank of servers (e.g., in a “cloud”). In various embodiments, the system 200 may further comprise at least one server and/or at least one database (not depicted) configured to communicate with one or more components of the system 200.

In some embodiments, a transceiver may be configured to communicate with compatible devices and ID tags when they are within a predetermined range. The transceiver may be compatible with one or more of: radio-frequency identification (RFID), near-field communication (NFC), Bluetooth™, low-energy Bluetooth™ (BLE), WiFi™, ZigBee™ ambient backscatter communications (ABC) protocols or similar technologies.

In example embodiments of the disclosed technology, the computing environments 206 and/or at least one controller 220 may include any number of hardware and/or software applications that are executed to facilitate any of the operations. The one or more I/O interfaces may be utilized to receive or collect data and/or user instructions from a wide variety of input devices. Received data may be processed by one or more computer processors as desired in various implementations of the disclosed technology and/or stored in one or more memory devices.

In various embodiments, the at least one acoustic transducer 202 may transmit at acoustic energy configured to cause the one or more bubbles to oscillate. In various embodiments, a radius of the one or more bubbles may expand and/or contract due to the emitted acoustic energy (e.g., ultrasound wave and/or the like). In various embodiments, the oscillation (e.g., expansion and/or contraction of the radius) of the one or more bubbles may cause at least one acoustic emission (AE). In various embodiments, the at least one receiver 206 of the system 200 may be configured to receive the AE from the one or more oscillating bubbles. The system 200 may use at least one ML model and/or controller monitor the AE data of the one or more bubbles to determine at least one acoustic emission level of the one or more bubbles. In other embodiments, the ML model may be separate from the controller. The ML model may predict at least one acoustic emission level that may indicate the collapse of the one or more bubbles. In various embodiments, the controller and/or ML model may cause the at least one acoustic transducer 202 to emit acoustic energy at least partially towards the one or more bubbles based at least in part on a predicted acoustic emission level that may be indicative of the collapse of the one or more bubbles. For example, the transducer might emit acoustic energy at a level that ensures the one or more bubbles do not collapse, e.g., an energy level that is as high as possible without resulting in collapse. In some embodiments, however, the transducer might emit acoustic energy at a level to cause at least a portion of the bubbles to collapse. In various embodiments, the emitted acoustic energy may be maintained below the predicted acoustic emission level that would cause the one or more bubbles to collapse.

### Example 1.1. Training Dataset

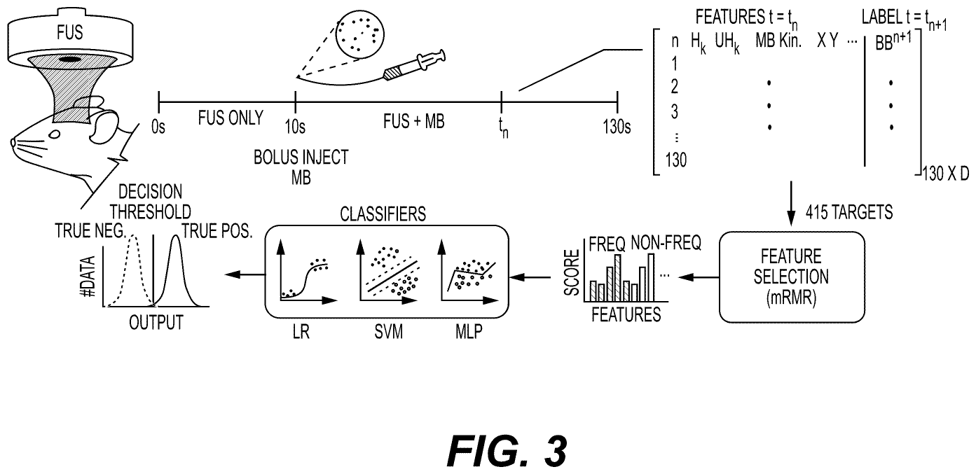

FIG. 3 illustrates an exemplary schematic for gathering at least one training data in accordance with various embodiments of the present disclosure. In various embodiments, at least one training dataset may comprise bubble acoustic emission data (e.g., microbubble acoustic emission data and/or the like) and/or may be used to train one or more machine learning (ML) models. The at least one training dataset may train the ML model to predict a threshold acoustic emission level for one or more bubbles (e.g., at least one acoustic emission level that would cause the one or more bubbles to collapse).

In various embodiments, an acoustic transducer may transmit acoustic energy configured to cause the one or more bubbles to oscillate in the body of the subject. The oscillation (e.g., expansion and/or contraction of the radius) of the one or more bubbles may cause an acoustic emission (AE). The AE can be one or more of a broadband emission, a harmonic emission, or a combination thereof. The onset of a broadband emission can indicate the collapse of one or more bubbles such that, the collapse of the one or more bubbles may cause damage to the tissue and/or vessel of the subject. Harmonic emission can indicate normal dynamics of the one or more bubbles.

As a non-limiting example, a training dataset may comprise microbubble acoustic emission (AE) data gathered from the blood-brain barrier (BBB) openings of 179 mice. The training dataset may comprise microbubble acoustic emission (AE) data gathered from 98,890 sonications with the same sonication parameters (500 kHz sonication frequency, 10 ms pulse length, and 1 Hz PRF). In various embodiments, the training dataset can consist of one or more other sonication parameters (e.g., frequency), which should be consistent throughout the dataset for proper training of ML model.

For example, one BBB opening session may consist of multiple sonication sessions, where each sonication session includes ˜130 sonication, each with 10 millisecond US pulse sent each second. For this exemplary training dataset, the label (e.g., an acoustic emission level that would cause the one or more microbubbles to collapse) at t=T should be a broadband occurrence at t=T+1.

### Example 1.2. Machine Learning (ML) Model Training

In various embodiments, the at least one training dataset comprising bubble acoustic emission (AE) data (e.g., microbubble acoustic emission data and/or the like) may be used to train at least one ML model. In various embodiments, the bubble AE data may comprise information regarding at least one feature. The at least one feature may be one or more of 2^{nd}-8^{th} harmonic levels, one or more of 2^{nd}-8^{th} ultra-harmonic levels, presence of at least one tumor, bubble kinetic level derived from normalized temporal harmonic level change, pulse number during sonication, at least one pressure, at least one presence of a disease, or a combination thereof. In various embodiments, the at least one feature may be arranged into a matrix (e.g., [98890×19] feature matrix) and/or used with a threshold acoustic emission level matrix (e.g., [98890×1] matrix) to train the at least one ML model.

In a non-limiting example embodiment, the feature matrix and/or the threshold acoustic emission level matrix may be used to train at least one ML model such that, the at least one ML model may be neural network classifier multilayer perceptron (MLP) model in MATLAB by utilizing the built-in ML toolbox functions. In various embodiments, the MLP model may predict at least one acoustic emission level indicative of the collapse of one or more bubbles (e.g., broadband emission) at T+1 using AE information at T, with T being the current pulse.

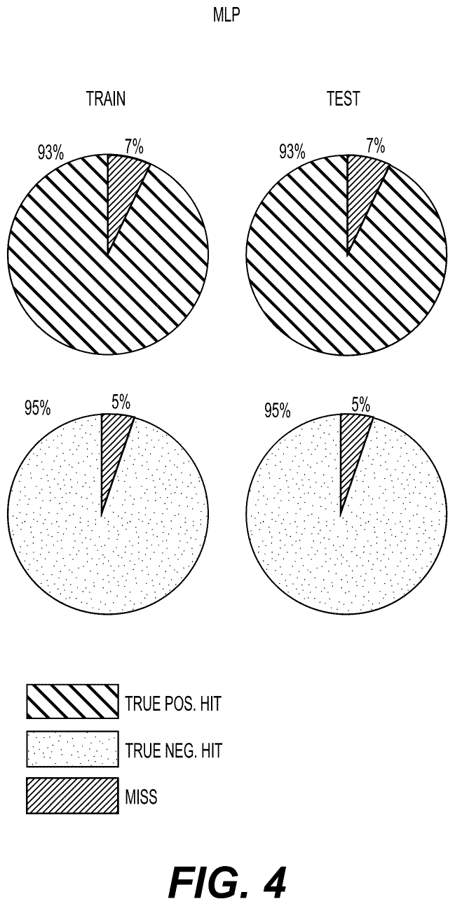

In one or more embodiments, the at least one ML model may be one or more of one or more of a classification model, a regression model, a support vector machine model, a logistic regression model, a different neural network model, a deep learning model, a multi-layer perceptron model, or a combination thereof. In a non-limiting example, the at least one ML model may be multi-layer perceptron (MLP) model and be trained with the dataset, which provided high accuracy in training/testing accuracy (93%) for prediction of broadband emissions higher than 6 dB re noise (depicted in FIG. 4).

### Example 1.3. Multilayer Perceptron (MLP) Model Training

In a non-limiting example, the machine learning (ML) model may be a MLP model. The MLP model may be designed and/or trained to identify and/or predict at least one relationship between current sonication data (e.g., current bubble acoustic emission data) and a future acoustic emission level that would be indicative of the collapse of at least one bubble (e.g., broadband emission). The MLP model may be trained using at least one selected feature (extracted from current sonication data) and/or at least one selected label (presence of broadband emission). Using the training dataset (described above), one or more of 2^{nd}-8^{th} harmonic levels, one or more of ultra-harmonic levels, 2^{nd}-8th harmonic levels, presence of at least one tumor, bubble kinetic level derived from normalized temporal harmonic level change, target region in the brain, pulse number during sonication, at least one pressure, at least one presence of a disease, or a combination thereof may be extracted as the at least one feature, depicted in Table 1. The MLP model may be trained to identify one or more of the selected features' (from current bubble acoustic emission data) relationship with the presence of at least one acoustic emission level that would cause the one or more bubbles to collapse (e.g., broadband emission and/or >6 dB re noise) at the following pulse. As depicted in FIG. 4, the trained MLP model was able to classify the onset of “future” acoustic emission level that would cause the one or more bubbles to collapse (e.g., that would be indicative of the collapse of the one or more bubbles, broadband emission, and/or the like) with accuracy of 93% in training dataset and 95% in test dataset. This demonstrates the potential of ML algorithm to provide an additional safety layer when in communication with a controller (e.g., closed-loop controller and/or open-loop controller).

TABLE 1
Features and Labels For MLP Model Training

| | | | | | | Label |
|---|---|---|---|---|---|---|
| Feature (t = T) | | | | | | (t = T + 1) |
| 2-8^{th} | 2-8^{th} Ultra- | Pressure | Target | MB | Tumor | Broadband |
| Harmonic | harmonic | | Location | Kinetics | Presence | emission >6 dB |

### Example 1.4 Shapley Additive Explanation Analysis (SHAP)

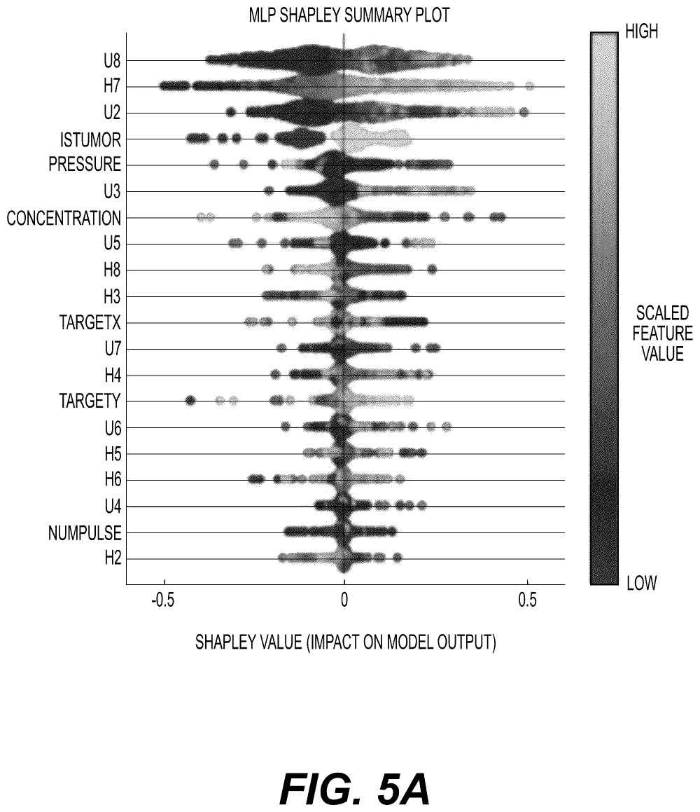

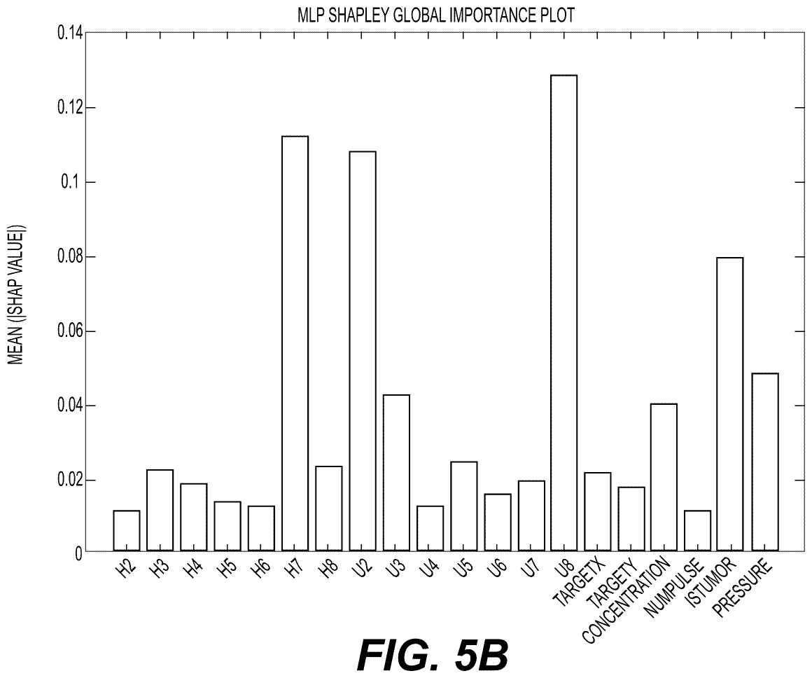

FIGS. 5A-5B graphically illustrates SHAP analysis on an exemplary training dataset in accordance with various embodiments of the present disclosure. FIG. 5A illustrates SHAP analysis onto an exemplary MLP model. Colors darker may indicate higher values of those features (e.g., 1-17 indicates higher H7, etc.) and/or lighter colors may indicate lower values. SHAP value (x-axis) represents the effect of each feature onto the ML model. Higher SHAP value may indicate higher impact on positive decision of the ML model and/or lower SHAP value may indicate higher impact on negative decision of the ML model. FIG. 5B illustrates SHAP global importance. Each bar represents the absolute value, as seen in FIG. 5A

To interpret the at least one trained ML model, SHAP analysis may be applied. As depicted in FIGS. 5A-5B, it may be found that the top features that drives at least one ML model to predicting at least one acoustic emission level that would cause the one or more bubbles to collapse (e.g., broadband emission prediction) may be the 2^{nd} and 8^{th} ultra-harmonic, 7^{th} harmonic, presence of tumor, and temporal bubble kinetics. These features provide support for avoiding onset of ultra-harmonic emissions, which is known as a strong indicator of upcoming broadband emission, and the features provides data-driven emphasis on the features that may be correlated with MB oscillation and collapse.

For instance, the ML model may consider the 7^{th} harmonic (7f₀) and 8^{th} ultra-harmonic (7.5f₀) levels with utmost importance is probably because they are in fact the frequencies close to the transducer frequency (e.g., PCD transducer center frequency of 3.5 MHz). Moreover, while 2^{nd} ultra-harmonic level may be outside of the sensitivity of the ML model, the ML model might have it highlighted since 2^{nd} ultra-harmonics is the strongest yet most unique signature of bubble dynamics (among feature frequencies) considering that other harmonic signals could be present from non-linearity in wave propagation. Importantly, an exemplary ML model may additionally suggest that pressure, bubble kinetics, and presence of tumor may have high impact on possibility of future acoustic emission levels that would cause the one or more bubbles to collapse (e.g., broadband emission). This, according to the ML model, indicates that the possibility of future acoustic emission level that would cause the one or more bubbles to collapse (e.g., broadband emission) may be in a close relationship with excitation parameter (pressure) and/or bubble concentration in the acoustic focus due to either different vascular structure (more dilated vessels in presence tumor) or temporal bubble kinetics in the body.

### Example 2. Integration of the Machine Learning (ML) Model with the Controller

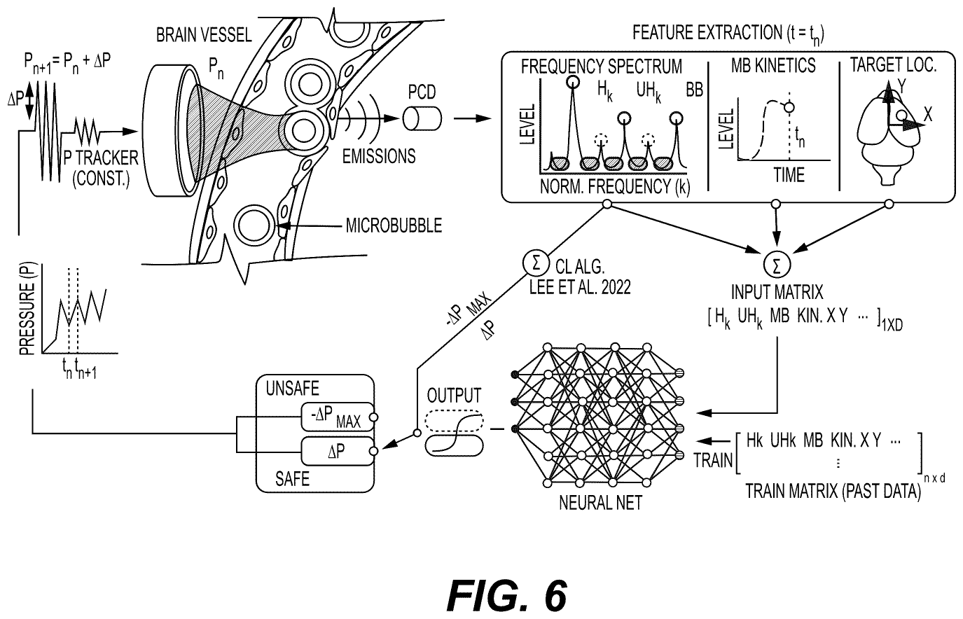

FIG. 6 illustrates an exemplary machine learning (ML) model integrated with (e.g., in communication with) a controller in accordance with various embodiments of the present disclosure. In one or more embodiments, the at least one ML model may be in communication with the controller before being trained with at least one training dataset. In other embodiments, the at least one ML model may be trained before being in communication with the controller. In various embodiments, the at least one trained ML model may be in communication with the controller (e.g., saved into a memory component of the controller) such that, the at least one ML model can predict acoustic emission levels of the one or more bubbles in real-time. The at least one ML model may communicate with the controller such that, the controller may cause an acoustic transducer to reduce the emitted acoustic energy to prevent one or more bubbles from collapsing. In various embodiments, the at least one ML model may cause the acoustic transducer to adjust the emitted acoustic energy to adjust one or more of a peak negative pressure, an acoustic intensity, a pulse repetition frequency (PRF), a pulse duration, a pulse length, a duty cycle, number of pulses, a waveform shape, or a combination thereof.

As a non-limiting example, a trained ML model may be in communication with a controller (e.g., MATLAB controller). The ML model can function as a last safety layer after all of controller's pressure decision. For instance, if the ML model predicts that a future acoustic emission level of the one or more bubbles is greater than threshold acoustic emission level for the one or more bubbles (e.g., at least one acoustic emission level that would be indicative of the collapse of the one or more bubbles, broadband emission, and/or the like), the ML model may cause the controller to react (i.e., reduce pressure) in a similar manner as if actual broadband event has occurred, instead of controlling harmonic level.

In a non-limiting example, at least one MLP model may be in communication with the controller such that, the at least one MLP model may predict at least one acoustic emission level that would cause the one or more bubbles to collapse based on acoustic emission levels of one or more bubbles in a body of a subject. For example, if the MLP model predicts a future acoustic emission level that indicates a possible collapse of the one or more bubbles (e.g., future broadband emission), the controller, in real-time, may react (i.e., reduce pressure) in a similar manner as if the collapse of the one or more bubbles has occurred, instead of controlling harmonic level.

### Example 3.1 Performance of Machine Learning (ML) Model with Closed Loop Controller

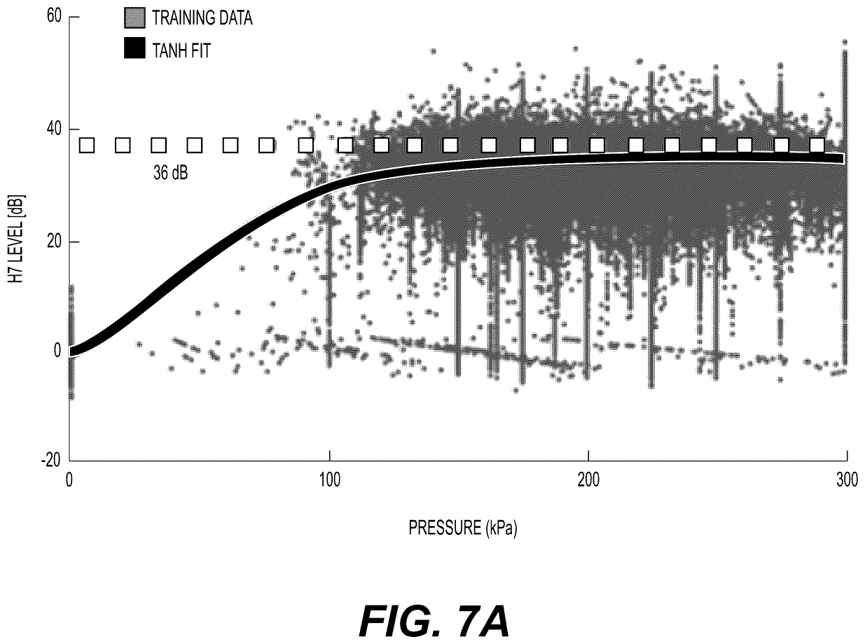

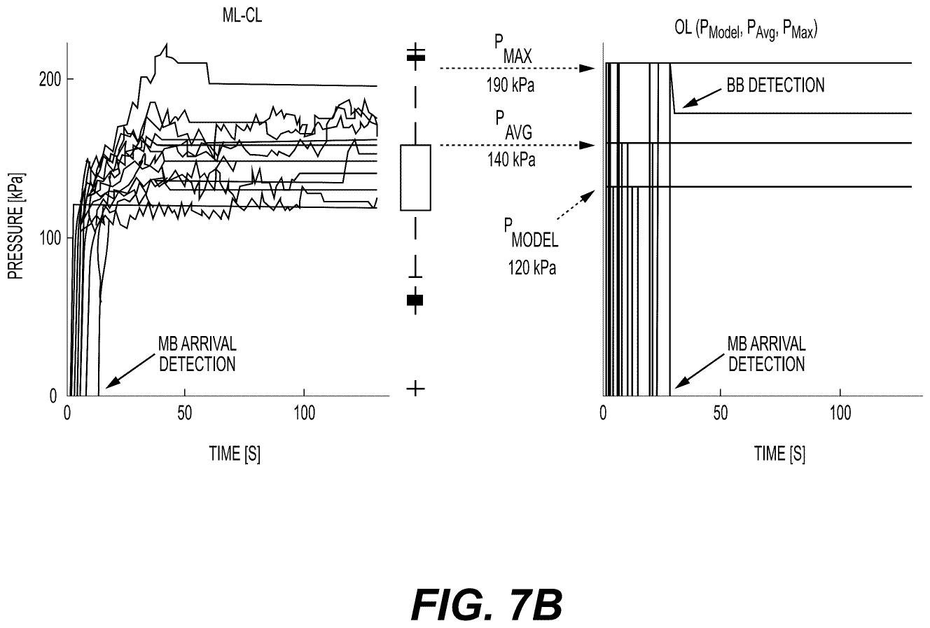

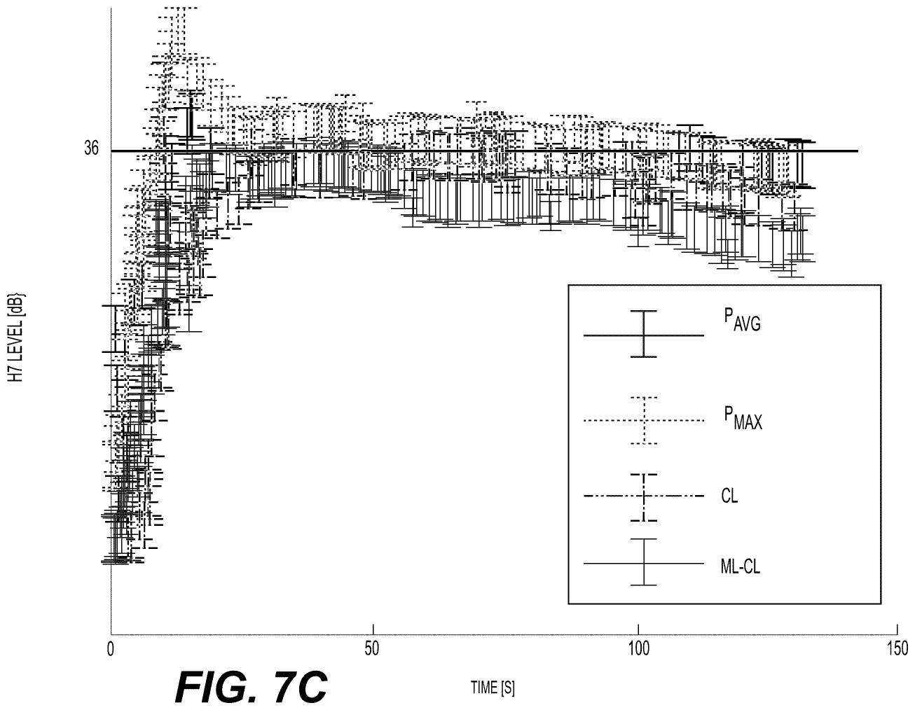

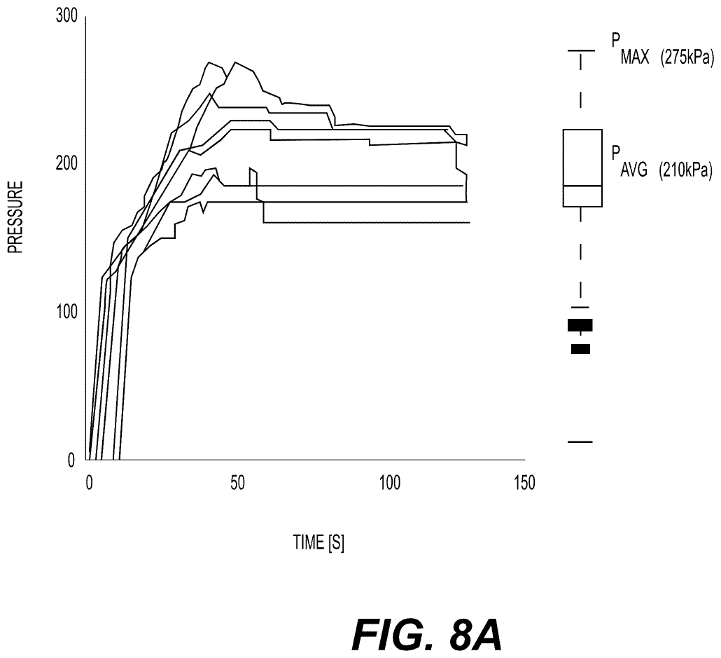

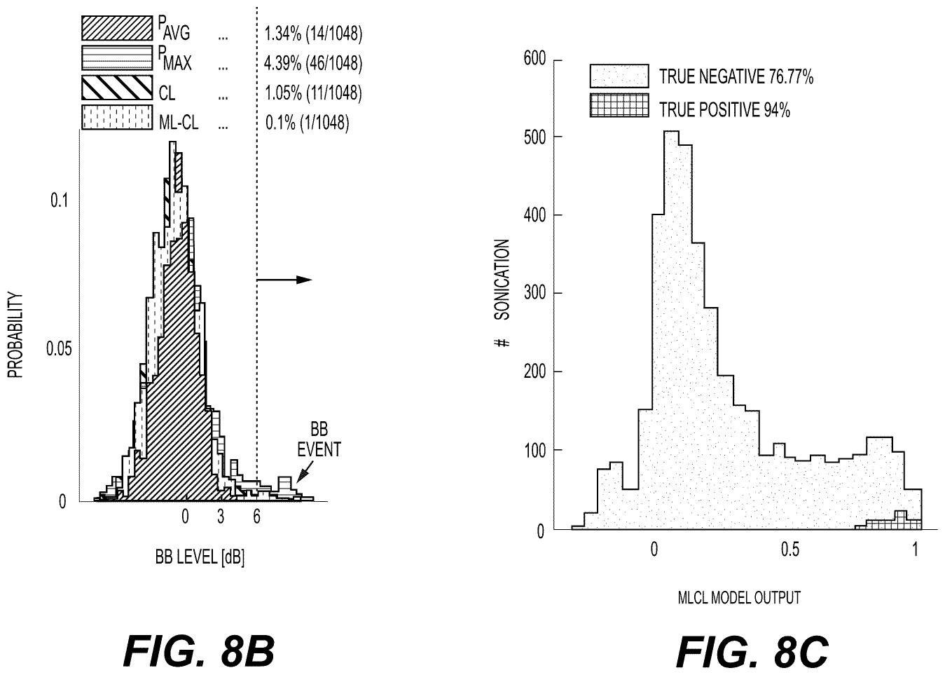

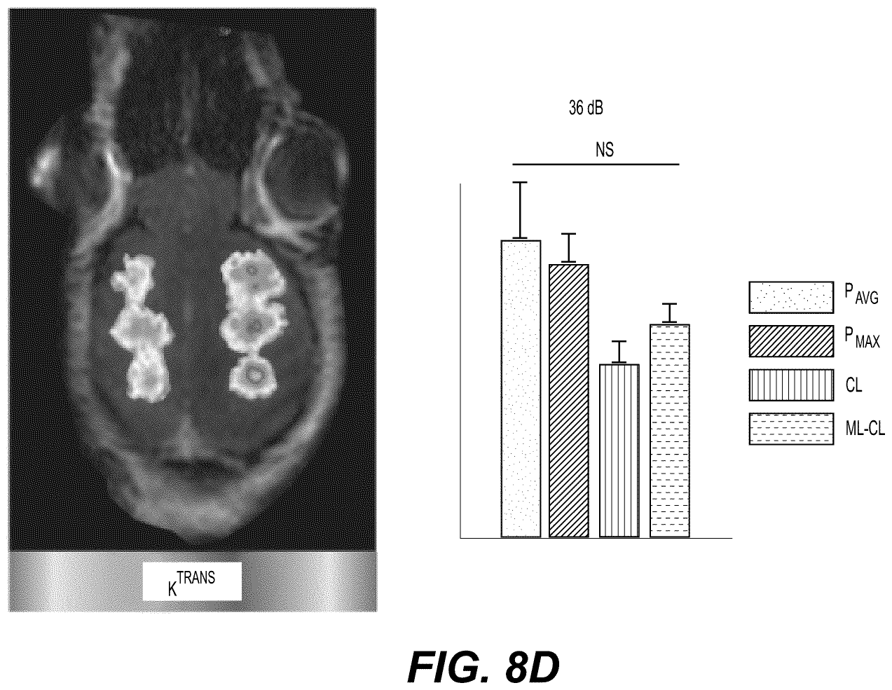

FIGS. 7A-7B graphically illustrate an exemplary cavitation threshold model acquired from a training dataset and machine learning closed loop (ML-CL) pressure decisions in accordance with various embodiments of the present disclosure. In various embodiments, the ML model may identify at least one target harmonic acoustic emission level to be achieved. The at least one target acoustic emission level may be selected based on at least a cavitation threshold model formed from the at least one training dataset (depicted in FIG. 7A). To test the ML model's ability to prevent the collapse of one or more bubbles, an extremely high target acoustic emission level was selected. In an exemplary embodiment, the extremely high target acoustic emission level may be 36 dB (ideally unachievable according to the exemplary cavitation threshold model).

Using the extremely high target acoustic emission level, the ML model was able to achieve target acoustic emission level of 36 dB, depicted in FIG. 7C. In a non-limiting exemplary embodiment, the ML model may be in communication with the controller. The ML model in communication with the controller was compared to its performance with other open-loop controllers (P_{avg} and Pₘₐₓ, pressures of 210 kPa and 275 kPa, respectively). These pressures were identified from the ML model in communication with the controller's pressure decisions, which are constant pressure controllers that starts sonication with detection of bubbles arrival to the brain and that reduces pressure when broadband is detected.

As a result, the ML model in communication with the controller may be able to reduce onset of broadband emissions (i.e., one or more bubbles from collapsing) down to 9.5% of that from previous work (closed-loop controller with reactive to broadband feature). Additionally, occurrences of acoustic emission levels that would cause the one or more bubbles to collapse (e.g., broadband emission) may be reduced down to 2.1% and 7.1% of that from open-loop controllers that utilized 275 kPa and 210 kPa, respectively. Importantly, the MLP model suggested that 94% of the acoustic emission levels that caused the one or more bubbles to collapse (e.g., broadband emissions) in other controllers (the reactive controllers) could have been prevented if the MLP model was integrated on the algorithms. Further, the reduction in acoustic emission levels that would cause the one or more bubbles to collapse (e.g., broadband emission events) may be done without compromising the efficiency of BBB opening evidenced by statistically insignificant difference in the Kₜᵣₐₙₛ, which is a permeability measurement.

In various embodiments, the ML model in communication with the controller can be applied to any application of MB-FUS to improve the safety, such as drug delivery (e.g., nanoparticles, genes, siRNA, chemotherapy, antibody, CART cell, and etc.) for neurological diseases (e.g., brain cancer, Alzheimer's, and Parkinsons), and treatment/tumor monitoring through biomarker assessment such as liquid biopsy. In one or more embodiments, controlling bubble dynamics may be used for one or more of ultrasound imagining, therapeutic treatment, therapeutic treatment for a tumor, liquid biopsy, drug delivery, increasing a permeability of a blood-brain barrier, maximizing treatment efficiency, or a combination thereof.

### Example 3.2 Performance of Machine Learning (ML) Model with Closed Loop Controller

In a non-limiting exemplary embodiment, to further evaluate the performance of an exemplary ML-assisted closed-loop controller (ML-CL) (e.g., ML model in communication with the controller), it may be crucial to establish an equitable comparison between ML-CL and currently available controller algorithms (e.g., conventional closed-loop controller, termed CL, and open-loop controller, termed OL). As depicted in Table 2, a comparison of established frameworks for OL, CL, and ML-CL algorithms may be summarized.

TABLE 2
Control Algorithms For Comparison

| Algorithm | | | Safety |
|---|---|---|---|
| Name | Pressure | Control Law | Feature |
| ML-CL | Varying | Closed-loop + MLP | Reaction + |
| | | | Prevention |
| CL | Varying | Closed-loop | Reaction |
| | | (Chapter 2&3) | |
| OL | ML-CL's Max Pressure | Reduce 5% pressure | Reaction |
| (P_{Max}) | (P_{Max}) | upon broadband | |
| | | emission | |
| OL | ML-CL's Average Pressure | Reduce 5% pressure | Reaction |
| (P_{Avg}) | (P_{Avg}) | upon broadband | |
| | | emission | |
| OL | Pressure from cavitation | Reduce 5% pressure | Reaction |
| (P_{Model}) | threshold model | upon broadband | |
| | (P_{Model}) | emission | |

In addition to its application on an exemplary ML model described above, past microbubble focused ultrasound AE dataset can also provide a reliable target level selection for closed-loop controllers through cavitation threshold model (depicted in FIG. 7A). An exemplary cavitation threshold can be extracted for 0.5 MHz excitation frequency to identify a target AE level to be used for the machine learning model in communication with the controller (ML-CL). As depicted in FIG. 7A, the relatively low AE level of 32 dB may be at the linear yet strong regime on the cavitation threshold model, which is thus applicable to an exemplary controller's target acoustic emission level. In a non-limiting example, the selected 32 dB target level may be selected on the cavitation threshold model to identify an “expected” pressure (120 kPa) to achieve the 32 dB target level, termed P_{model} hereafter, which can be used for OL. Thus, by integrating these insights from AE dataset, one or more exposure conditions may be established for the ML model in communication with the controller (ML-CL and CL with 32 dB target level) and open-loop controllers (OL using 120 kPa, i.e., P_{model}) for further performance evaluation and comparison. Consequently, using the 32 dB target level, one or more additional pressures were identified and used for OL algorithms described in Table 2 (P_{Avg} and P_{Max}) from BBB opening in rodents using ML-CL algorithm, which were 140 kPa and 190 kPa, respectively (depicted in FIG. 7B).

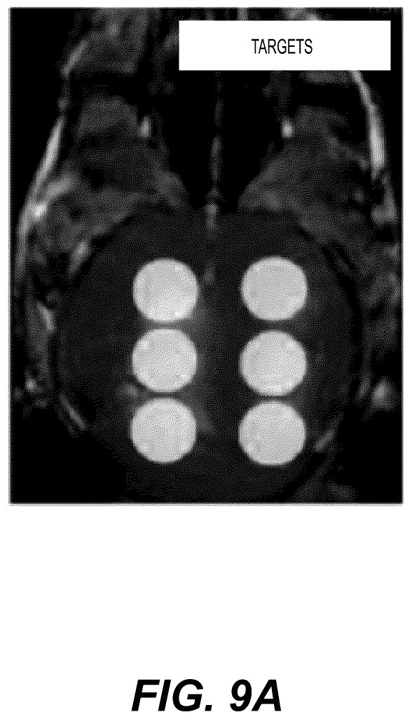

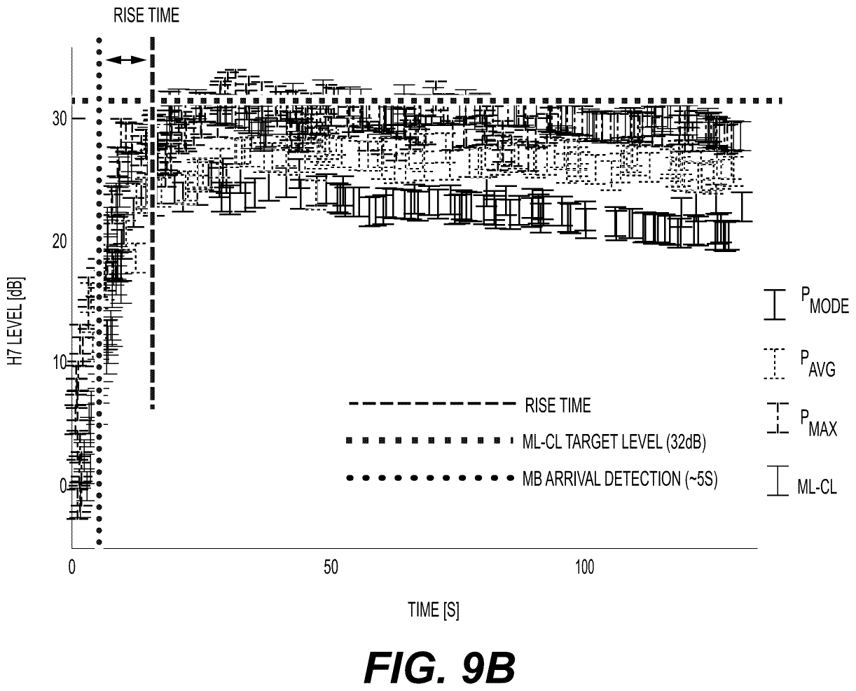

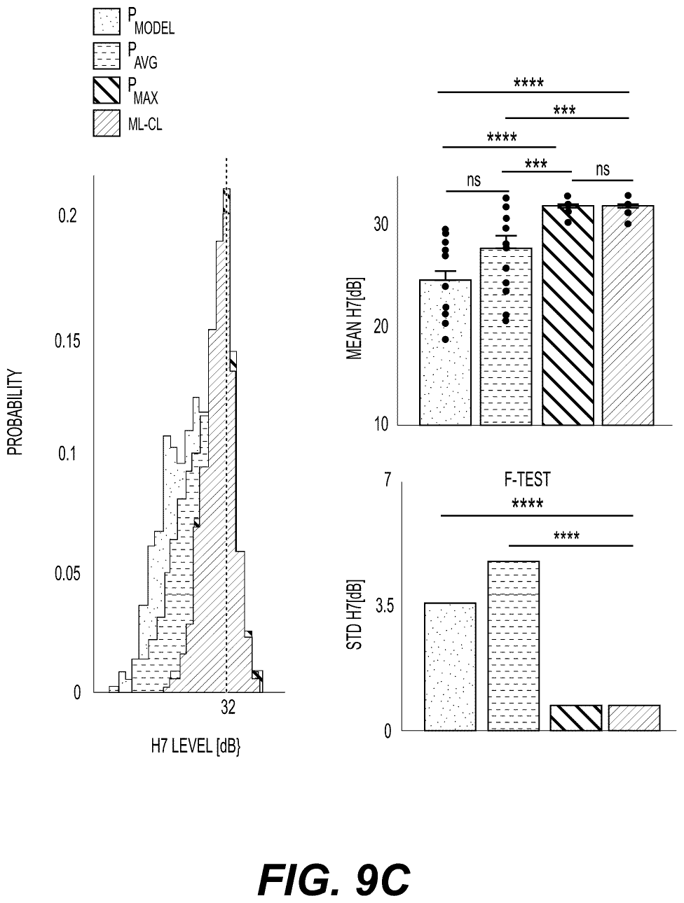

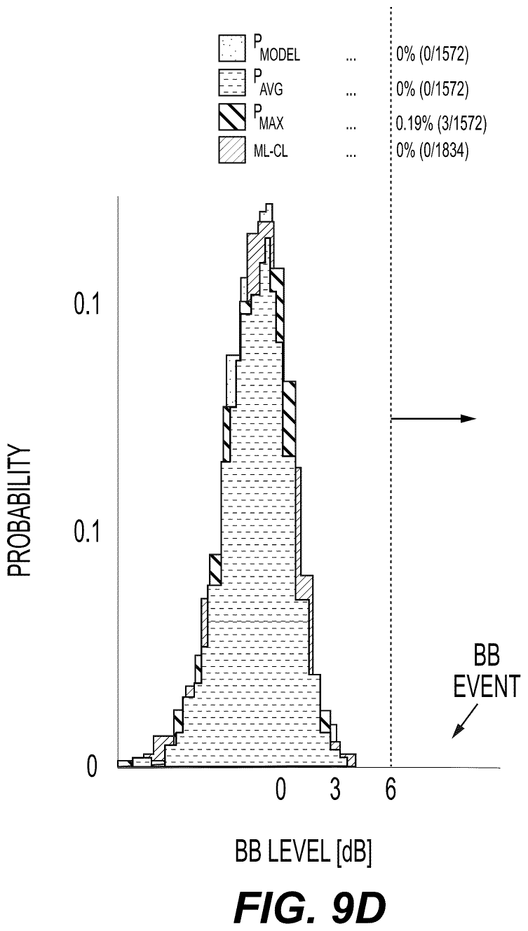

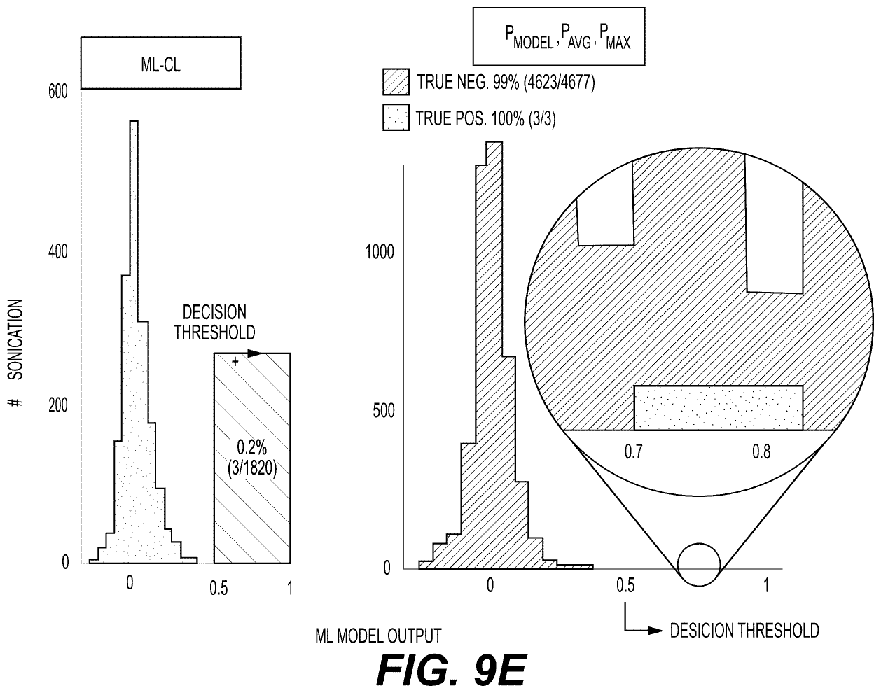

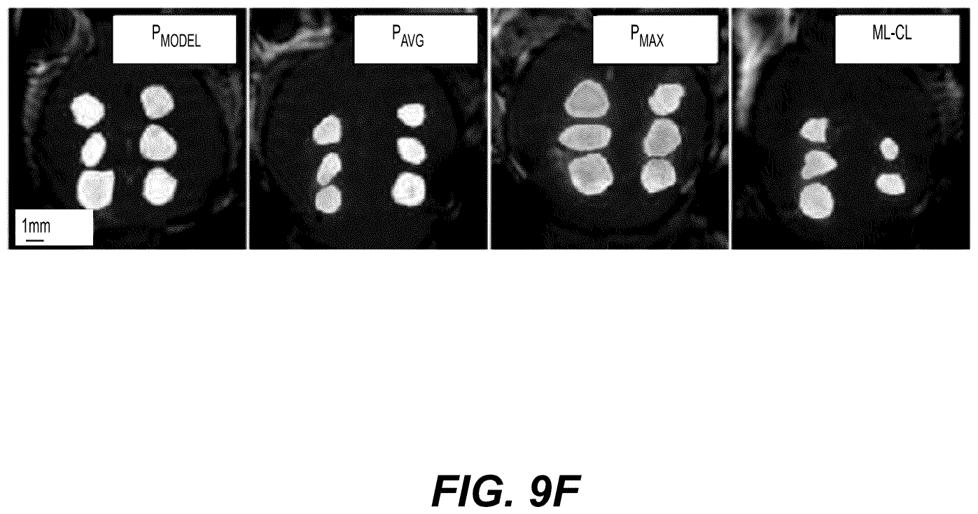

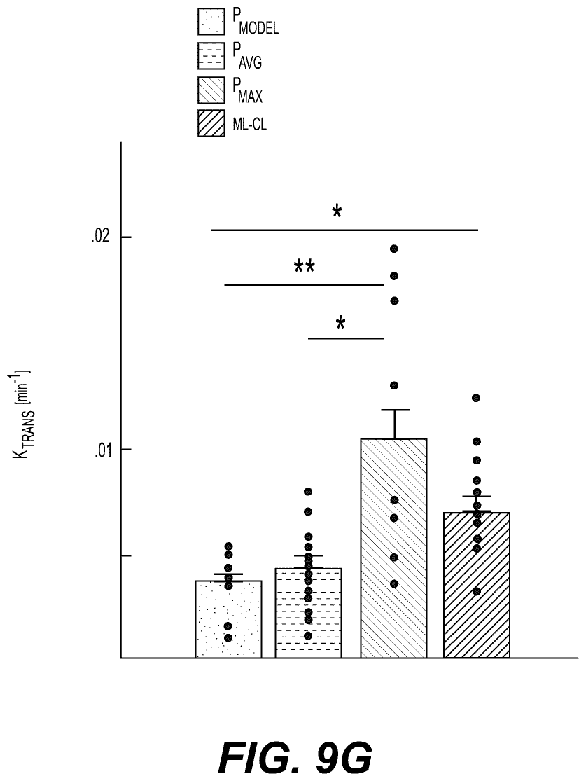

In a non-limiting exemplary embodiment, an exemplary ML model in communication with a controller was used at 6-target locations in healthy mice (depicted in FIG. 9A). It was found that the exemplary ML model in communication with a controller (e.g., ML-CL controller) may be able to achieve 31.6±0.6 dB 7^{th} harmonic level (depicted in FIGS. 9B-9C), which was significantly higher than OL controllers that utilized P_{model} and P_{Avg}. The OL controller averaged harmonic level lower than 30 dB (p<0.0001 and p<0.01, respectively). In various embodiments, the exemplary ML model in communication with a controller (e.g., ML-CL controller) may produce significantly lower deviation in harmonic levels evidenced by f-test compared to P_{Model} and P_{Avg} groups, which in aggregate suggests the necessity of closed-loop algorithm in fine tuning of strong bubble dynamics. While the OL controller using Pₘₐₓ has performed similarly as the ML-CL, the OL was found to produce broadband emission events (e.g., the one or more bubbles collapsing) (depicted in FIG. 9D).

In various embodiments, despite achieving strong AE levels, the exemplary ML model in communication with a controller (e.g., ML-CL controller) did not produce any >6 dB acoustic emission levels that would cause the one or more bubbles to collapse (e.g., broadband emission events) due to the ML model's predictions made during sonication and the consequent reduction of FUS exposure (3 instances out of 1820 sonication, depicted in FIG. 9E). To further assess an exemplary ML model's prediction ability, the exemplary ML model was applied onto the AE data acquired from P_{model} and P_{Avg} and Pₘₐₓ groups. The exemplary ML model predicted that 100% (3/3) of the acoustic emission levels that caused the one or more bubbles to collapse (e.g., broadband emission instances) could have been prevented upon its integration onto the control algorithms (depicted in FIG. 9E).

In various embodiment, to assess the impact of the controllers to increase BBB permeability, the Kₜᵣₐₙₛ values were quantified from each controller group (depicted in FIG. 9F). The Kₜᵣₐₙₛ values quantified indicated that the exemplary ML model in communication with the controller resulted in significantly higher permeability compared to P_{Model} group (p<0.05) at an increasing trend amongst P_{Model} and P_{Avg} (depicted in FIG. 9G). While the permeability change from the P_{Max} algorithm had the maximum effect, as was observed previously, it was at the cost of broadband emission events which is highly unfavorable due to risks for cumulative tissue damage when MB-FUS enhanced liquid biopsy is used repeatedly for diagnosis or treatment monitoring.

### Example 4. Exemplary Controller Used in Liquid Biopsy

In a non-limiting embodiment, an exemplary ML model in communication with a controller may be applied onto GL261 bearing mice for safe and effective liquid biopsy. The cell line was transfected to express Gaussia luciferase (G-Luc) protein, which is a bioluminescent molecule that is secreted by the transfected cell. G-Luc gene and protein may be used as test molecules to assess liquid biopsy. When the tumor reached 2.5 mm in diameter (measured with MRI), each mouse was treated with 5 sonication targets separated by 1.5 mm to cover the tumor region. Blood samples were collected retro-orbitally 5 minutes before and after treatment, and 2 hours-post-treatment using an EDTA-coated collection straw attached to a non-coated 1.5 ml microcentrifuge tube. Samples were allowed to coagulate in ice for 10 min prior to 1,000 g centrifugation for 20 min. Serum was allocated for protein and DNA, G-Luc gene, and protein quantifications in the blood

### Example 5. Exemplary Controller Employing the Present Methods

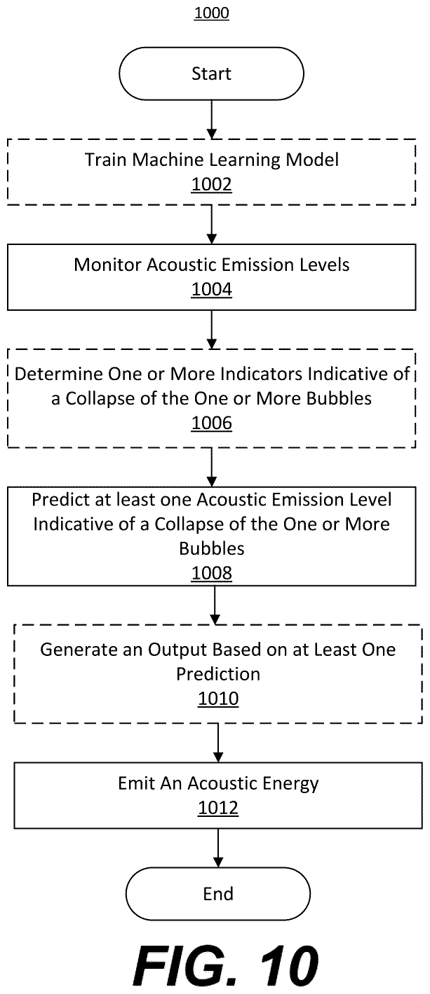

FIG. 10 illustrates a flow diagram of an exemplary method 1000 for controlling bubble dynamics of the one or more bubbles in accordance with various embodiments of the present disclosure. The steps of method 1000 may be performed by one or more components of the system, computing environment, and/or controller, as described in more detail with respect to FIGS. 1-3 and 6.

Method 1000 may optionally begin with training at least one ML model with the at least one training dataset, Block 1002. In various embodiments, the at least one training dataset may comprise bubble acoustic emission data, as described above.

In Block 1004, in various embodiments, the system and/or ML model alone and/or in communication with the controller may monitor acoustic emission levels of one or more bubbles in a body of a subject. In various embodiments, the at least one acoustic transducer (e.g., waveform generator) may provide acoustic energy (e.g., initial acoustic energy, first acoustic energy, and/or the like) through an outer surface of the vessel and/or tissue and to at least a portion of the one or more bubbles. In various embodiments, the acoustic energy may cause the one or more bubbles to oscillate. In one or more embodiments, a frequency at which the one or more bubbles oscillate may be dependent on one or more of a bubble size, gas properties of a bubble, surrounding fluid, transmitted frequency from the waveform generator, or a combination thereof. In one or more embodiments, the one or more bubbles may comprise an oscillation motion that is one or more of a linear oscillation, nonlinear oscillation, or a combination thereof. In various embodiments, the oscillation of the one or more bubbles may cause the one or more bubbles to emit at least one acoustic wave (e.g., acoustic emission level).

In optional Block 1006, in various embodiments, the ML model may be configured to determine at least one indicator related to the acoustic emission levels of the one or more bubbles (e.g., acoustic emission levels of the one or more bubbles oscillating). The one or more indicators may be indicative of the collapse of at least one bubble. In various embodiments, the one or more indicators may be one or more of at least one bubble kinetic, an average pressure, a maximum pressure, a minimum pressure, at least one microbubble kinetic, a brain region, presence of at least one tumor, or a combination thereof. In various embodiments, the at least one receiver may be in communication with the ML model and/or controller to monitor the AE data of the one or more bubbles in real-time.

In Block 1008, in various embodiments, the ML model may predict at least one acoustic emission level that may be indicative of the collapse of at least one bubbles. In various embodiments, the predicted acoustic emission level that would cause the one or more bubbles to collapse may be based on at least the oscillation of the one or more bubbles and/or the acoustic emission levels of the one or more bubbles in a body of a subject. In various embodiments, the predicted acoustic emission level may be based at least in part on the AE data of the one or more bubbles and/or the one or more indicators.

In optional Block 1010, in various embodiments, the ML model and/or the controller may generate at least one output based on the at least one prediction from the ML model. In one or more embodiments, the at least one output may be a real-time advisory instruction configured to guide therapeutic sonication operated at a constant pressure. In other embodiments, the at least one output may be a real-time advisory instruction configured to adjust acoustic energy delivered by an acoustic transducer operated with controller.

In Block 1012, in various embodiment, the ML model and/or the controller may cause the acoustic transducer to emit an acoustic energy to the one or more bubbles based at least in part on the predicted acoustic emission level that would cause the one or more bubbles to collapse. In one or more embodiments, the ML model and/or the controller may cause the acoustic transducer to emit an acoustic energy that adjust one or more of a peak negative pressure, an acoustic intensity, a pulse repetition frequency (PRF), a pulse duration, a pulse length, a duty cycle, a number of pulses, a waveform shape, or a combination thereof.

In this description, numerous specific details have been set forth. It is to be understood, however, that implementations of the disclosed technology can be practiced without these specific details. In other instances, well-known methods, structures and techniques have not been shown in detail in order not to obscure an understanding of this description. References to “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described can include a particular feature, structure, or characteristic, but not every implementation necessarily includes the particular feature, structure, or characteristic. Further, repeated use of the phrase “in one implementation” does not necessarily refer to the same implementation, although it can.

As used in this application, the terms “component,” “module,” “system,” “server,” “processor,” “memory,” and the like are intended to include one or more computer-related units, such as but not limited to hardware, firmware, a combination of hardware and software, software, or software in execution. For example, a component may be, but is not limited to being, a process running on a processor, an object, an executable, a thread of execution, a program, and/or a computer. By way of illustration, both an application running on a computing device and the computing device can be a component. One or more components can reside within a process and/or thread of execution and a component may be localized on one computer and/or distributed between two or more computers. In addition, these components can execute from various computer readable media having various data structures stored thereon. The components may communicate by way of local and/or remote processes such as in accordance with a signal having one or more data packets, such as data from one component interacting with another component in a local system, distributed system, and/or across a network such as the Internet with other systems by way of the signal.

Certain embodiments and implementations of the disclosed technology are described above with reference to block and flow diagrams of systems and methods and/or computer program products according to example embodiments or implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, may be repeated, or may not necessarily need to be performed at all, according to some embodiments or implementations of the disclosed technology.

These computer-executable program instructions may be loaded onto a general-purpose computer, a special-purpose computer, a processor, or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks.

As an example, embodiments or implementations of the disclosed technology may provide for a computer program product, including a computer-usable medium having a computer-readable program code or program instructions embodied therein, said computer-readable program code adapted to be executed to implement one or more functions specified in the flow diagram block or blocks. Likewise, the computer program instructions may be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

The features and other aspects and principles of the disclosed embodiments may be implemented in various environments. Such environments and related applications may be specifically constructed for performing the various processes and operations of the disclosed embodiments or they may include a general-purpose computer or computing platform selectively activated or reconfigured by program code to provide the necessary functionality. Further, the processes disclosed herein may be implemented by a suitable combination of hardware, software, and/or firmware. For example, the disclosed embodiments may implement general purpose machines configured to execute software programs that perform processes consistent with the disclosed embodiments. Alternatively, the disclosed embodiments may implement a specialized apparatus or system configured to execute software programs that perform processes consistent with the disclosed embodiments. Furthermore, although some disclosed embodiments may be implemented by general purpose machines as computer processing instructions, all or a portion of the functionality of the disclosed embodiments may be implemented instead in dedicated electronics hardware.

The disclosed embodiments also relate to tangible and non-transitory computer readable media that include program instructions or program code that, when executed by one or more processors, perform one or more computer-implemented operations. The program instructions or program code may include specially designed and constructed instructions or code, and/or instructions and code well-known and available to those having ordinary skill in the computer software arts. For example, the disclosed embodiments may execute high level and/or low-level software instructions, such as machine code (e.g., such as that produced by a compiler) and/or high-level code that can be executed by a processor using an interpreter.

The technology disclosed herein typically involves a high-level design effort to construct a computational system that can appropriately process unpredictable data. Mathematical algorithms may be used as building blocks for a framework, however certain implementations of the system may autonomously learn their own operation parameters, achieving better results, higher accuracy, fewer errors, fewer crashes, and greater speed.

This written description uses examples to disclose certain embodiments of the disclosed technology, including the best mode, and also to enable any person skilled in the art to practice certain embodiments of the disclosed technology, including making and using any devices or systems and performing any incorporated methods. The patentable scope of certain embodiments of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A controller for predicting and controlling of bubble dynamics in real-time, the controller comprising:
one or more processors;
a machine learning model in communication with the controller, wherein the machine learning model is trained based on bubble acoustic emission data;
at least one memory in communication with the controller and the machine learning model and storing computer program code that, when executed by the controller, is configured to cause the controller to:
monitor acoustic emission levels of one or more bubbles in a body of a subject;
predict, via the machine learning model, at least one acoustic emission level indicative of a collapse of the one or more bubbles; and
cause, in real-time, an acoustic transducer to emit acoustic energy to the one or more bubbles based at least in part on the at least one predicted acoustic emission level indicative of the collapse of the one or more bubbles.

2. The controller of claim 1, wherein:
the controller is one or more of a constant pressure sonication controller, an open-loop controller configured to adjust acoustic energy by a preset control law, a reactive controller configured to adjust acoustic energy upon detection of the collapse of the one or more bubbles, a closed-loop controller configured to dynamically adjust acoustic energy based on real-time feedback, or a combination thereof;
the machine learning model is one or more of a classification model, a regression model, a support vector machine model, a logistic regression model, a different neural network model, a deep learning model, a multi-layer perceptron model, or a combination thereof; and
the one or more bubbles is one or more of microbubbles, ultrasound contrast microbubbles, nanobubbles, cavitation nuclei, bubbles generated in situ during focused ultrasound (FUS) intervention, or a combination thereof.

3. The controller of claim 2, wherein:
the bubbles acoustic emission data comprises at least one feature arranged in a matrix; and
the at least one feature arranged in the matrix comprises one or more of 2^{nd}-8^{th} harmonic levels, one or more of 2^{nd}-8^{th} ultra-harmonic levels, presence of at least one tumor, bubble kinetic level derived from normalized temporal harmonic level change, pulse number during sonication, at least one pressure, at least one presence of a disease, or a combination thereof.

4. The controller of claim 2, wherein preventing the collapse of the one or more bubbles prevents damage to at least a portion of at least one vessel and/or at least one tissue of the subject.

5. The controller of claim 1, wherein:
an emitted acoustic energy is maintained below the at least one predicted acoustic emission level that is indicative of the collapse of the one or more bubbles; and
adjusting the acoustic energy comprises adjusting one or more of a peak negative pressure, an acoustic intensity, a pulse repetition frequency (PRF), a pulse duration, a pulse length, a duty cycle, a number of pulses, a waveform shape, or a combination thereof.

6. The controller of claim 1, wherein the at least one memory further comprises computer program code that, when executed by the controller, is configured to cause the controller to:
generate at least one output based on at least one prediction from the machine learning model, wherein the at least one output is a real-time advisory instruction configured to guide therapeutic sonication operated at a constant pressure, or a real-time advisory instruction configured to adjust acoustic energy delivered by an acoustic transducer operated with controller.

7. The controller of claim 1, wherein predicting and controlling bubble dynamics is used for one or more of ultrasound imaging, therapeutic treatment, therapeutic treatment for a tumor, liquid biopsy, drug delivery, increasing a permeability of a blood-brain barrier, maximizing treatment efficiency, histotripsy, gene delivery, other FUS-related inventions including but not limited to neuromodulation, ablation, or immunomodulation, or a combination thereof.

8. The controller of claim 1, wherein the at least one memory further comprises computer program code that, when executed by the controller, is configured to cause the controller to:
determine, using the machine learning model, one or more indicators of a collapse of the one or more bubbles, the one or more indicators is one or more of at least one bubble kinetic, an average pressure, a maximum pressure, a minimum pressure, at least one microbubble kinetic, a brain region, presence of at least one tumor, or a combination thereof, wherein the one or more indicators indicates an acoustic emission level indicative of the collapse of the one or more bubbles.

9. A non-transitory computer readable medium having stored thereon instructions for predicting and controlling of bubble dynamics, which when executed by one or more processors in communication with an acoustic transducer, causes the processors to:
monitor acoustic emission levels of one or more bubbles in a body of a subject;
predict, based on bubble acoustic emission data, at least one acoustic emission level indicative of a collapse of one or more; and
cause, in real-time, the acoustic transducer to emit acoustic energy to the one or more bubbles based at least in part on the at least one predicted acoustic emission level indictive of the collapse of the one or more bubbles.

10. The non-transitory computer readable medium of claim 9, wherein:
the non-transitory computer readable medium is a portion of a controller;
the controller is one or more of a constant pressure sonication controller, an open-loop controller configured to adjust acoustic energy by a preset control law, a reactive controller configured to adjust acoustic energy upon detection of the collapse of the one or more bubbles, a closed-loop controller configured to dynamically adjust acoustic energy based on real-time feedback, or a combination thereof; and
the one or more bubbles is one or more of microbubbles, ultrasound contrast microbubbles, nanobubbles, cavitation nuclei, bubbles generated in situ during focused ultrasound (FUS) intervention, or a combination thereof.

11. The non-transitory computer readable medium of claim 10, wherein:
the bubble acoustic emission data comprises at least one feature arranged in a matrix; and
the at least one feature arranged in the matrix comprises one or more of 2^{nd}-8^{th} harmonic levels, one or more of 2^{nd}-8^{th} ultra-harmonic levels, presence of at least one tumor, microbubble kinetic level derived from normalized temporal harmonic level change, pulse number during sonication, at least one pressure, at least one presence of a disease, or a combination thereof.

12. The non-transitory computer readable medium of claim 10, wherein preventing the collapse of the one or more bubbles prevents damage to at least a portion of at least one vessel and/or at least one tissue of the subject.

13. The non-transitory computer readable medium of claim 9, wherein:
an emitted acoustic energy is maintained below the at least one predicted acoustic emission level that is indicative of a collapse of the one or more bubbles; and
adjusting the acoustic energy comprises adjusting one or more of a peak negative pressure, an acoustic intensity, a pulse repetition frequency (PRF), a pulse duration, a pulse length, a duty cycle, a number of pulses, a waveform shape, or a combination thereof.

14. The non-transitory computer readable medium of claim 9, wherein the non-transitory computer readable medium further comprises instructions which when executed by one or more processors, further causes the processors to:
generate at least one output based on the at least one predicted acoustic emission level, wherein the at least one output is a real-time advisory instruction configured to guide therapeutic sonication operated at a constant pressure, or a real-time advisory instruction configured to adjust acoustic energy delivered by an acoustic transducer operated with controller.

15. The non-transitory computer readable medium of claim 9, wherein predicting and controlling bubble dynamics is used for one or more of ultrasound imaging, therapeutic treatment, therapeutic treatment for a tumor, liquid biopsy, drug delivery, increasing a permeability of a blood-brain barrier, maximizing treatment efficiency, histotripsy, gene delivery, other FUS-related inventions including but not limited to neuromodulation, ablation, or immunomodulation, or a combination thereof.

16. The non-transitory computer readable medium of claim 9, wherein the non-transitory computer readable medium further comprises instructions which when executed by one or more processors in communication with an acoustic transducer, further causes the processors to:
determine one or more indicators of a collapse of the one or more bubbles, the one or more indicators is one or more of at least one bubble kinetic, an average pressure, a maximum pressure, a minimum pressure, at least one microbubble kinetic, a brain region, presence of at least one tumor, or a combination thereof, wherein the one or more indicators indicates an acoustic emission level indicative of the collapse of the one or more bubbles.

17. A method for controlling bubble dynamics, the method comprising:
monitoring acoustic emission levels of one or more bubbles in a body of a subject;
predicting, via a machine learning model, at least one acoustic emission level that would indicate a collapse of the one or more bubbles; and
emitting an acoustic energy to the one or more bubbles based at least in part on the at least one predicted acoustic emission level that would indicate the collapse of the one or more bubbles, wherein the one or more bubbles is one or more of microbubbles, ultrasound contrast microbubbles, nanobubbles, cavitation nuclei, bubbles generated in situ during focused ultrasound (FUS) intervention, or a combination thereof.

18. The method of claim 17, further comprising:
training the machine learning model based at least on bubble acoustic emission data, wherein:
the machine learning model is one or more of a classification model, a regression model, a support vector machine model, a logistic regression model, a different neural network model, a deep learning model, a multi-layer perceptron model, or a combination thereof;
the bubbles acoustic emission data comprises at least one feature arranged in a matrix; and
the at least one feature arranged in the matrix comprises one or more of 2^{nd}-8^{th} harmonic levels, 2^{nd}-8^{th} ultra-harmonic levels, presence of at least one tumor, microbubble kinetic level derived from normalized temporal harmonic level change, pulse number during sonication, at least one pressure, at least one presence of a disease, or a combination thereof.

19. The method of claim 18, further comprising:
determining, using the machine learning model, one or more indicators of a collapse of the one or more bubbles, the one or more indicators is one or more of at least one bubble kinetic, an average pressure, a maximum pressure, a minimum pressure, at least one microbubble kinetic, a brain region, presence of at least one tumor, or a combination thereof, wherein the one or more indicators indicates an acoustic emission level indicative of the collapse of the one or more bubbles; and
generating at least one output based on the at least one prediction from the machine learning model, wherein the at least one output is a real-time advisory instruction configured to guide therapeutic sonication operated at a constant pressure, or a real-time advisory instruction configured to adjust acoustic energy delivered by an acoustic transducer operated with controller.

20. The method of claim 17, wherein controlling the bubble dynamics comprises using one or more of a constant pressure sonication controller, an open-loop controller configured to adjust acoustic energy by a preset control law, a reactive controller configured to adjust acoustic energy upon detection of the collapse of the one or more bubbles, a closed-loop controller configured to dynamically adjust acoustic energy based on real-time feedback, or a combination thereof.
